(19) United States
(12) Patent Application Publication
(10) Pub. No.: US 2006/0105469 A1
(43) Pub. Date: May 18, 2006

(54) Assay devices

(75) Inventors: Peter Lea, Toronto (CA); Michelle Gal, Toronto (CA); Nicole Szabados Haynes, Toronto (CA); Richard A. Prokopowicz, Toronto (CA)

Correspondence Address: DIMOCK STRATTON LLP, 20 QUEEN STREET WEST SUITE 3202, BOX 102, TORONTO, ON, M5H 3R3, CA

(73) Assignee: UMEDIK, INC., Toronto (CA)

(21) Appl. No.: 11/319,117

(22) Filed: Dec. 27, 2005

Related U.S. Application Data
(62) Division of application No. 09/866,305, filed on May 25, 2001, now abandoned.
Application No. 09/866,305 is a continuation-in-part of application No. PCT/US00/13056, filed on May 12, 2000.
Application No. PCT/US00/13056 is a continuation-in-part of application No. 09/335,732, filed on Jun. 18, 1999, now Pat. No. 6,403,384.

(51) Int. Cl. G01N 33/558

(52) U.S. Cl. 436/514

(57) ABSTRACT

Assay devices are disclosed comprising a base defining a cavity and an insert received in the cavity. The cavity has major surface and at least one sidewall, preferably surrounding the major surface. The insert comprises a first surface with a portion opposing the major surface of the cavity. A space is provided between the portion of the first surface and the major surface for the receipt of a fluid sample. The space has an entrance defined by the first surface of the insert and the major surface. The insert also comprises a second surface opposing the first surface and having an input portion for the application of a fluid sample. The input portion is in fluid communication with the entrance to the space, such that a fluid sample applied to the input portion passes to the entrance to the space and into the space. At least one or more passages is preferably defined through the insert, for passage of the fluid sample through the insert, to the entrance to the space. The second surface of the insert also comprises a reading portion for analyzing the fluid sample in the space. Reagents may be provided in the space for identifying and quantifying the presence of one or more analytes in the fluid sample. Preferably, the assay device is transparent. The portion of the first surface and the first surface of the insert and the major surface of the cavity may be separated by a distance effective to cause capillary flow of the fluid sample into the space from the entrance to the space.

### RELATED APPLICATIONS

The present application is a continuation in part of PCT/US00/13056, filed on May 12, 2000, which is a continuation-in-part of U.S. Ser. No. ______ (to be assigned) (Attorney Docket Number 254/112), filed on May 16, 2001, which is a national phase applications based on PCT/CA99/01079, filed on Nov. 12, 1999, which are both continuation in parts of U.S. Ser. No. 09/335,732, which was filed on Jun. 18, 1999. These application are assigned to the assignee of the present invention and are incorporated by reference herein, in their entireties.

### FIELD OF THE INVENTION

The present invention is an assay device for identifying the presence or absence of an analyte in a fluid sample. A quantitative measurement of the concentration or amount of the analyte in the fluid sample may also be obtained. The assay device may include a filter for separating unwanted components of the fluid sample greater than a predetermined size from the fluid components of the sample.

### BACKGROUND OF THE INVENTION

Toxic bacteria account for several million cases of food-related illnesses and 9,000 deaths per year in the United States alone. Contaminated poultry and meat products are a major cause of these deaths and illness. The four most common pathogens infecting poultry and meat products are E. coli O157:H7, campylobacter jejuni/coli, salmonella and listeria monocytogenes. Assays for detecting these and other microorganisms require that the samples be cultured. The pathogens are typically detected by culturing a food sample on an agar plate. Multiple culturing steps are usually required, after which the plate may be sent to a laboratory for analysis. It could take several days to obtain results. Paper test strips including test reagents such as antibodies, are also used. However, paper strip tests have low sensitivity.

Contamination of water supplies also causes illness and death. The United States Environmental Protection Agency has determined that the level of E. coli in a water supply is a good indicator of health risk. Other common indicators are total coloforms, fecal coloforms, fecal streptococci and enterococci. Water samples are currently analyzed for these microorganisms with membrane filtration or multiple-tube fermentation techniques. Both types of tests are costly and time consuming and require significant handling. They are not; therefore, suitable for field-testing.

Many disease conditions, such as bacterial and viral infections, many cancers, heart attacks and strokes, for example, may be detected through the testing of blood and other body fluids, such as saliva, urine, semen and feces for markers that have been shown to be associated with particular conditions. Early and rapid diagnosis may be the key to successful treatment. Standard medical tests for quantifying markers, such as ELISA-type assays, are time consuming and require relatively large volumes of fluid.

The accurate and rapid detection and measurement of microorganisms, such as bacteria, viruses, fungi or other infectious organisms and indicators in food and water, on surfaces where food is prepared, on other surfaces which should meet sanitary standards is, therefore, a pressing need in biological samples. There is also a serious need for the accurate and rapid identification of microorganisms and markers of the health of a patient.

Testing fluid from biological samples, food products or water supplies, for example, often requires that cellular or other extraneous, non-fluid materials, be removed from the sample. In testing blood, for example, it is typically necessary to separate the blood cells (erythrocytes, leukocytes and platelets) from the plasma. When taking fluid samples from a stream, a host of materials, such as dirt, plant fragments, pebbles, and fish and animal feces are typically included in the sample. In the prior art, chromatographic paper, fiberglass or other fibrous materials have been provided in assay devices to wick the fluid component of a sample from the cellular or other such components, prior to testing. Use of such fibrous materials may reduce the rate and volume of fluid flow through the assay device, increasing the time required to obtain the test results. When health concerns require that test results be obtained as soon as possible, such delays are not acceptable. Centrifugation of the test samples has also been used to separate non-fluid components. Centrifugation, however, requires cumbersome equipment, making it inappropriate for field-testing. Relatively large volumes of fluid are also typically required. Particularly when dealing with human or animal fluids, it is preferred to withdraw a minimum amount of fluid from a patient.

In a typical test assay, a fluid sample is mixed with a reagent, such as an antibody, specific to a particular analyte (the substance being tested for), such as an antigen. The reaction of the analyte with the reagent may result in a color change that may be visually observed, or in chemiluminescent, bioluminescent or fluorescent species that may be observed with a microscope or detected by a photodetecting device, such as a spectrophotometer or photomultiplier tube. The reagent may also be a fluorescent or other such detectable labeled reagent that binds to the analyte. Radiation scattered, reflected, transmitted or absorbed by the fluid sample may also be indicative of the identity and type of analyte in the fluid sample.

In a commonly used assay technique, two types of antibodies are used, both specific to the analyte. One type of antibody is immobilized on a solid support. The other type of antibody is labeled by conjugation with a detectable marker and mixed with the sample. A complex between the first antibody, the substance being tested for and the second antibody is formed, immobilizing the marker. The marker may be an enzyme, or a fluorescent or radioactive marker, which may then be detected. See, for example, U.S. Pat. No. 5,610,077.

There are presently many examples of one-step assays and assay devices for detecting analytes in fluids. One common type of assay is the chromatographic assay, wherein a fluid sample is exposed to a chromatographic strip containing reagents. A reaction between a particular analyte and the reagent causes a color change on the strip, indicating the presence of the analyte. In a pregnancy test device, for example, a urine sample is brought into contact with a test pad comprising a bibulous chromatographic strip containing reagents capable of reacting with and/or binding to human chorionic gonadotropin (“HCG”). The urine sample moves by capillary flow along the bibulous chromatography strips. The reaction typically generates a color change, which indicates that HCG is present. While the presence of a quantity of an analyte above a threshold may be determined, the actual amount or concentration of the analyte is unknown.

In order to quantitatively measure the concentration of an analyte in a sample and to compare test results, it is advantageous to use a consistent test volume of the fluid sample each time the assay is performed. The analyte measurement may then be assessed without having to adjust for varying volumes. U.S. Pat. No. 4,088,448, entitled “Apparatus for Sampling, Mixing the Sample with a Reagent and Making Particularly Optical Analysis”, discloses a cuvette with two planar surfaces defining a cavity of predetermined volume for receiving a sample fluid. The fluid is drawn into the cavity by capillary force, gravity or a vacuum. The sample mixes with a reagent in the cavity. The sample is then analyzed optically. There is no convenient location for placement of the sample on the disclosed device. The open side of the cavity is brought into contact with the sample, possibly by dipping the open side into the sample. There is also no separation medium incorporated in the device. If separation is required, it must take place prior to drawing the sample into the device.

In U.S. Pat. No. 4,978,503, entitled “Devices for Use in Chemical Procedures”, a device is shown including upper and lower transparent plates fixed together in parallel, opposed and spaced relation by adhesive to form a capillary cell cavity. The cavity is open at opposite ends. One open end is adjacent to a platform portion of the lower plate for receiving the sample. The other open end allows for the exit of air. Immobilized test reagents are provided within the cavity, on inner surfaces of one or both plates. The reaction between the sample and the reagent may be detected optically, from one of the open ends of the cavity. Filter paper may be provided on the platform to restrict the passage of red blood cells into the cavity, for testing blood. In one embodiment, the plates are supported by plastic arms. Removable handles are also provided for use during various stages of the use of the device. The disclosed devices appear to be complex to manufacture and use.

U.S. Pat. No. 6,197,494 B1, entitled “Apparatus for Performing. Assays on Liquid Samples Accurately, Rapidly and Simply”, discloses assay devices comprising a base, an overlay defining a receiving opening, a reaction space and a conduit connecting the opening to the space, and a cover also defining a sample receiving opening and a viewing opening. When assembled, the sample receiving openings are aligned and the viewing opening is positioned over the reaction space. Heat sealing, solventbonding or other appropriate techniques may be used to connect the layers to each other. Light may be provided through any of the layers, which act as waveguides, for optical analysis of the sample. By providing the light through the edge of the overlay, for example, light scattered, transmitted or absorbed by the sample may be detected by appropriate placement of standard detectors. By providing the light through the base or cover, fluorescence of the sample may be detected. Light may pass through the reaction space transverse to the layers, as well. Light passing through the reaction space may also be reflected off a layer, back through the reaction space. The disclosed devices comprise at least three pieces which require assembly. A simpler device would be desirable.

### SUMMARY OF THE INVENTION

The present invention is an assay device for identifying the presence or absence of an analyte in a fluid sample. A quantitative measure of the detected analyte may also be made. The analyte may be a microorganism, such as a bacteria, a virus or a fungus. The analyte may also be a protein, an enzyme, an antibody, an antigen, an immunoglobulin, a drug, a hormone or a chemical, for example. Multiple analytes may be identified in the same assay device. The fluid sample may be a human or animal sample, such as blood, urine or feces. The fluid sample may also be derived from food, water or soil, for example. The fluid sample may be a suspension. The analyte is identified and/or quantified by mixing the fluid sample with reagents, such as antibodies, specific to the analyte. Certain antibodies may be labeled with a detectable marker. The reagents may be provided in the assay device, may be added to the fluid sample prior to application to the assay device, or both. The results of a reaction between the fluid sample and test reagents can be analyzed visually with a microscope. Radiation, such as light, generated, reflected, scattered, absorbed or transmitted by the fluid sample may also be detected by a detector, such as a photoconductive detector. A photodiode, a photomultiplier tube or a CCD may be used, for example. A machine, such as a spectrophotometer, luminometer or fluorometer, may also be used. The detected radiation may be analyzed to determine the presence or absence of an analyte, and/or the amount or concentration of the analyte in the fluid sample.

In accordance with one embodiment of the present invention, an assay device is disclosed comprising a base defining a cavity and an insert received in the cavity. The cavity has major surface and at least one sidewall, preferably surrounding the major surface. The insert comprises a first surface with a portion opposing the major surface of the cavity. A space is provided between the portion of the first surface and the major surface for the receipt of a fluid sample. The space has an entrance defined by the first surface of the insert and the major surface. The insert also comprises a second surface opposing the first surface and having an input portion for the application of a fluid sample. The input portion is in fluid communication with the entrance to the space, such that a fluid sample applied to the input portion passes to the entrance to the space and into the space. At least one or more passages is preferably defined through the insert, for passage of the fluid sample through the insert, to the entrance to the space. The second surface of the insert also comprises a reading portion for analyzing the fluid sample in the space. Reagents may be provided in the space for identifying and quantifying the presence of one or more analytes in the fluid sample. Preferably, the assay device is transparent.

The portion of the first surface and the first surface of the insert and the major surface of the cavity may be separated by a distance effective to cause capillary flow of the fluid sample into the space from the entrance to the space.

A filtering medium is preferably provided in the passage through the insert. The filtering medium is preferably a plurality of particles which transiently abut each other when the fluid sample passes through the passages. The transiently abutting particles form transient interstitial passages which are effective to remove unwanted material greater than a predetermined size, from the fluid sample. The particles are preferably supported in the passage by a porous material. The porous material may be a nylon or polyester mesh, a polycarbonate film or a polysulfane membrane, for example. Reagents may be provided among the particles in addition to or instead of the reagents in the space.

The insert is preferably press-fit into the cavity. Protrusions are preferably provided on either the edge of the insert or the interior surface of the sidewall of the cavity, or both, to provide the press-fit, thereby reducing stress on the insert. The insert and the side wall of the cavity preferably have portions or vents which are spaced a greater distance from each other than the distance between the second surface of the insert and the major surface of the cavity, so that less capillary force is generated between the insert and the sidewall, than in the space. Air may escape from the space through the vents. In addition, the fluid sample evenly fills the space, instead of rapidly moving along the edge of the insert. Air bubbles in the space are thereby minimized.

A lid is preferably provided, slideably engaging the base. The lid may be selectively moved over the input portion after a fluid sample is applied, to protect the fluid sample from contamination. In addition, the lid maintains humidity in the region around the fluid sample, slowing evaporation of the fluid sample.

A wall is preferably provided around the input portion to also protect the fluid sample and to prevent the fluid sample from being applied to or to moving onto the reading portion.

Opposing reflective surfaces may be provided on opposite sides of the space to cause multiple reflections of radiation, such as light, introduced into the space, to increase the optical path length for spectrophotometric measurements. The reflective surfaces may be supported by opposing interior surfaces of the space or the opposing portions of the reading portion and the base, outside of the space.

### BRIEF DESCRIPTION OF THE DRAWINGS

FIG. 1 is a perspective view of the assay device in accordance with one embodiment of the present invention;

FIG. 2 is an exploded view of the assay device of FIG. 1;

FIG. 3 is a top view of the assay device of FIG. 1;

FIG. 4 is a top view of the base of the assay device of FIG. 1;

FIG. 5 is a top view of the insert base of the assay device of FIG. 1;

FIG. 6 is a cross-sectional view of the insert, along line 6-6 in FIG. 5;

FIG. 7A is a cross-sectional view of the assay device, along line 7-7 of FIG. 1;

FIG. 7B is an enlarged view of a portion of FIG. 7A;

FIG. 8 is a cross-sectional view of FIG. 1 along line 8-8;

FIG. 9 is a cross-sectional view corresponding to FIG. 8, showing an alternative embodiment of the insert and the base;

FIG. 10 is a cross-sectional view of the assay device along line 10-10 in FIG. 1, showing the lid;

FIG. 11 is a cross-sectional view of an assay device in accordance with another embodiment of the invention, wherein the cavity is recessed in the base;

FIG. 12 is a cross-sectional view of an assay device in accordance with another embodiment of the invention, wherein the chamber for receiving the fluid sample is not a capillary chamber;

FIG. 13 is a perspective view of a multi-layer carrier for use with the embodiment of FIG. 12;

FIG. 14 is a cross-sectional view of a luminometer in accordance with another embodiment of the present invention;

FIG. 15 is a cross-sectional view of an assay device with a reflective surface, in accordance with another embodiment of the present invention;

FIG. 16 is a schematic representation of the assay device in accordance with another embodiment of the present invention, including two reflective surfaces;

FIG. 17 is a cross-sectional view of a the capillary chamber of the assay device of the present invention, including a pair of reflective surfaces;

FIG. 18 is a top view of the assay device of the present invention, showing the two reflective surfaces; and

FIG. 19 is a bottom view of the assay device of FIG. 18.

### DETAILED DESCRIPTION OF THE INVENTION

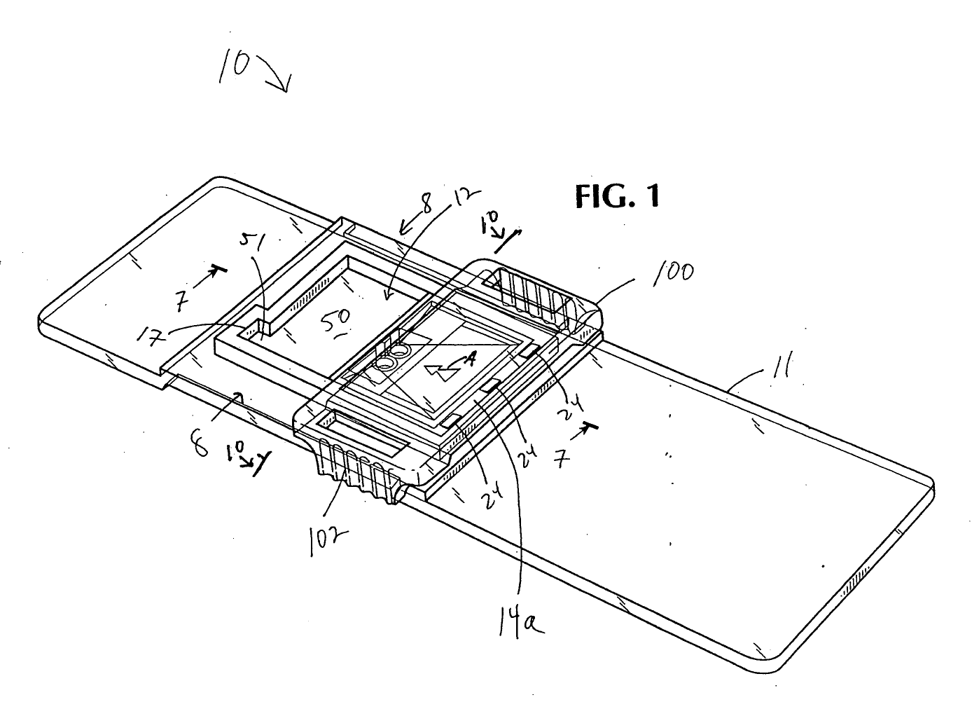

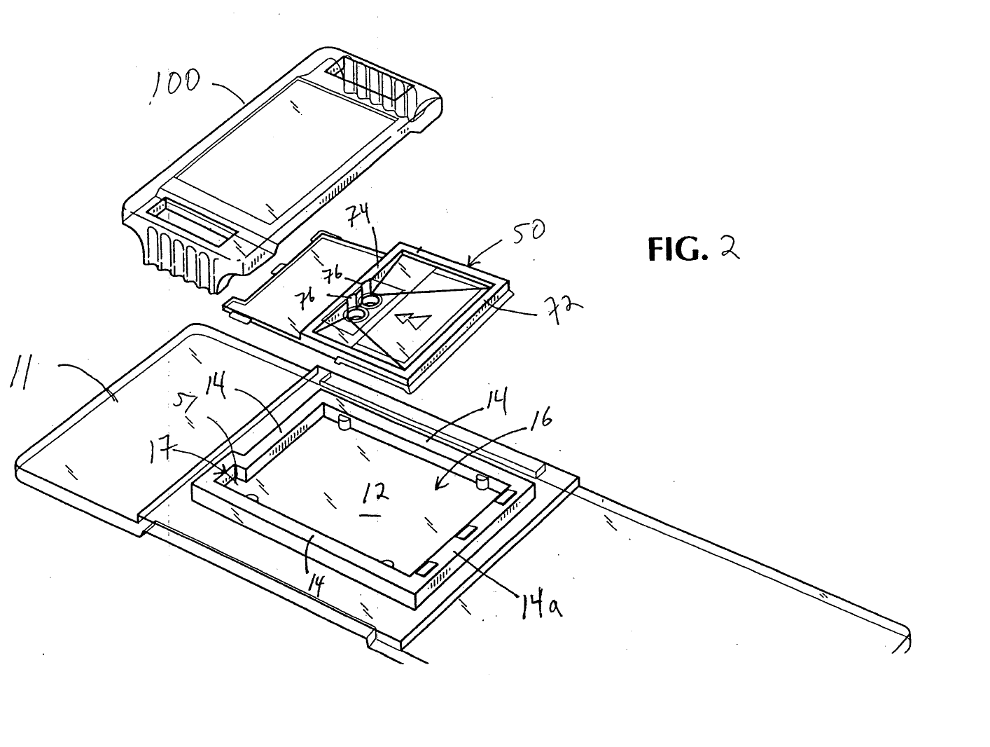

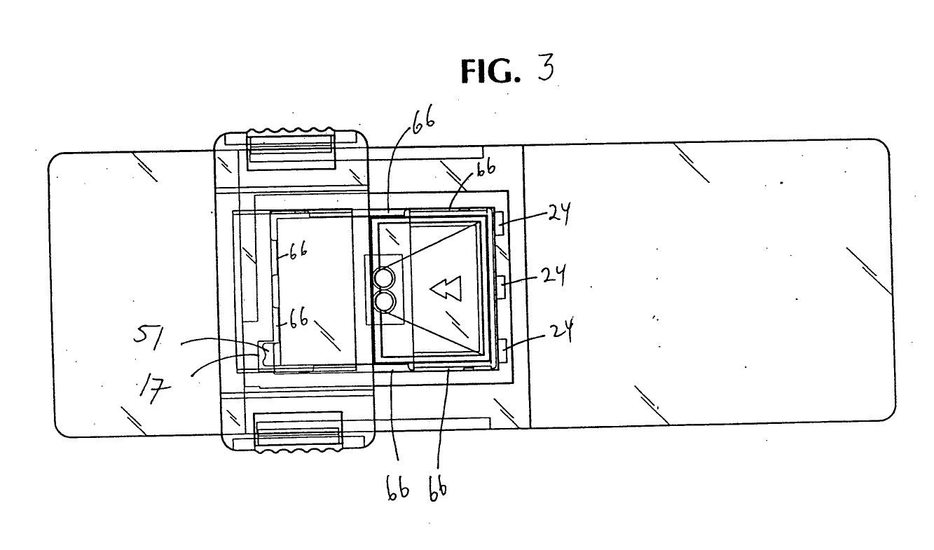

FIG. 1 is a perspective view of the assay device 10 in accordance with one embodiment of the present invention. The assay device 10 comprises three pieces: a base 11, an insert 50 within a cavity 12 defined by the base and a lid 100 snapped over the base. FIG. 2 is an exploded view of the assay device 10 of FIG. 1, more clearly showing the base 11, the cavity 12, the insert 50 and the lid 100. FIG. 3 is a top view of the assay device 10. Preferably, the base 11, the insert 50 and the lid 100 are molded in plastic. Preferred materials are discussed below.

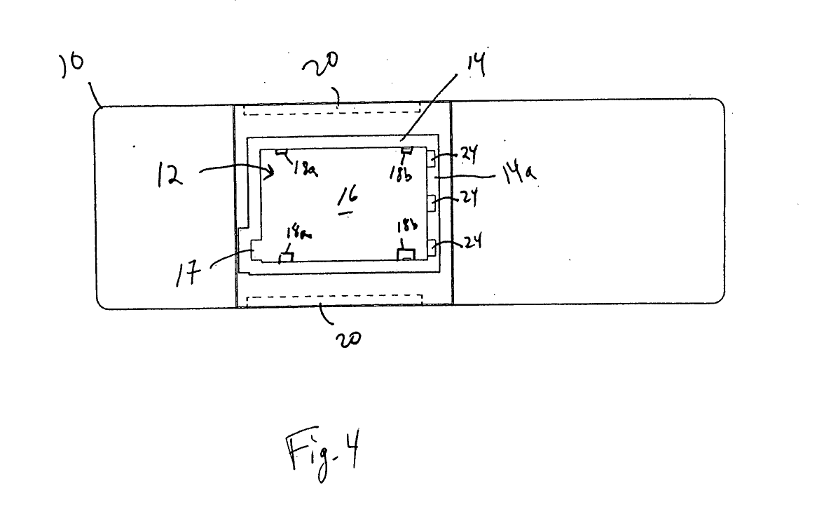

FIG. 4 is a top view of the base 11. As shown in FIGS. 1-4, the base 11 defines a cavity 12 with a sidewall 14 surrounding a major bottom wall 16. The cavity 12 has an open face opposite the major bottom wall 16. In this embodiment, the sidewall 14 comprises four walls connected to form a rectangle. The sidewall 14 may also define a circle, an oval, or any other convenient shape. The shape of the insert 50 preferably matches the shape of the cavity 12. The sidewalls 14 extend transverse to the base 11. Preferably, the sidewalls 14 are substantially perpendicular to the major wall 16, and extend above the surface of the base 11. In that case, to accommodate the well, the base 11 may need to be made thicker. Preferably, the base is molded as a single part. However, the base 11 may comprise multiple parts which are glued or otherwise assembled together.

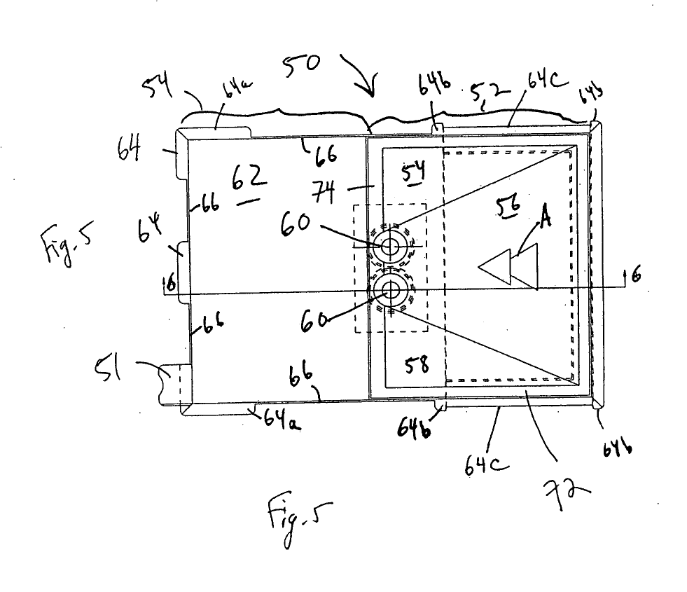

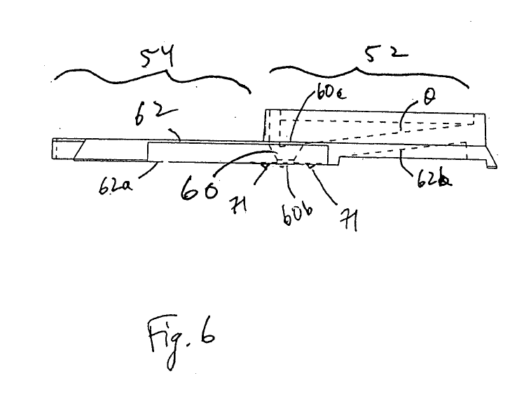

FIG. 5 is a top view of the insert 50. FIG. 6 is a cross-sectional view of the insert 50, along line 6-6 in FIG. 5. As shown in FIGS. 5 and 6, the insert 50 includes an input portion 52 for receiving a fluid sample for analysis and a reading portion 54 for viewing the results of a reaction of the sample with test reagents in the space between the insert 50 and the bottom wall 16 of the cavity 12. The reading portion 54 provides a large surface for analyzing the fluid sample beneath it.

The input portion 52 preferably comprises a tapered surface to guide a fluid sample towards one or more passages 60 through the insert 50. Preferably, three panels 54, 56, 58 are provided, tapered towards two openings 60 through the insert 50. An angle θ of about 8° from horizontal for the central panel 56 is sufficient for gravity to generate a resultant force on the fluid sample to draw the fluid sample toward the passages 60, as shown in FIG. 6. Two passages 60 are preferably provided, to increase the rate of fluid flow through the insert 50. Each passage 60 has an entrance 60a and an exit 60b. Each passage 60 is preferably inwardly tapered from the entrance 60a to the exit 60b. The passages 60 are separated by a distance of about 0.5 mm. An arrow “A” may be provided on the input portion, pointing towards the passages 60, to aid the user in identifying the input portion 52 and the passages 60.

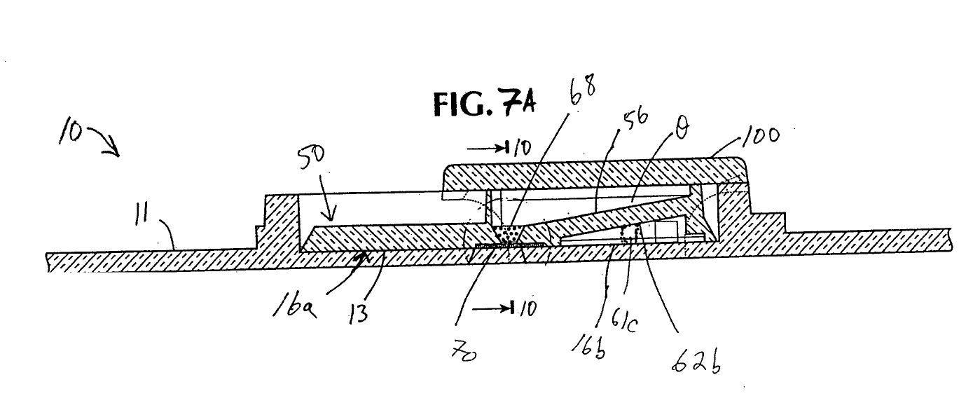

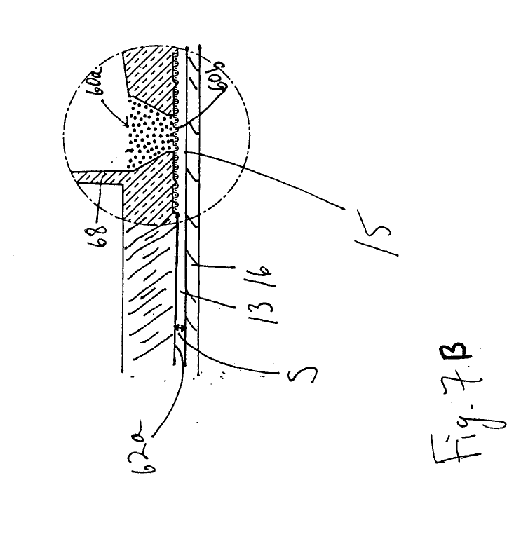

The reading portion 54 comprises a planar wall 62 with an underside surface 62a that is preferably parallel to an opposing region 16a of the major bottom wall 16 of the base 11 when the insert 50 is within the cavity 12. FIG. 7A is a cross-sectional view of the assay device 10, along line 7-7 of FIG. 1. FIG. 7B is an enlarged view of a portion of FIG. 7A, showing the space “S” between the underside 62a of the planar wall 62 and the bottom wall 16. The space S defines a chamber 13 with an entrance 15 proximate the exit of the passages 60, as shown in part in FIG. 7B. The space S and chamber 13 are not perceptible in FIG. 7. In one embodiment, the distance between the underside 62a and the bottom wall 16 is small enough to provide a capillary force on fluid brought into contact with the passages 60, for drawing the fluid between the underside 62a of the planar wall 62 and the bottom wall 16. The chamber 13 is therefore a capillary chamber 13.

To detect microorganisms and other markers in food, water and biological fluid samples, the distance between the region 16a of the bottom wall 16 and the underside 62a is preferably between about 10 microns to about 120 microns. The distance may be as small as 0.10 microns or less. A distance of up to about 250 microns may be provided, however, more time could be required to fill the larger capillary chamber 13. Capillary chambers 13 may be formed with the distance between the walls of up to about 2 millimeters, although the filling time would be even longer.

The insert 50 is preferably secured within the chamber 12 by a press-fit, which has been found to sufficiently secure the insert in the chamber 12 and to be simple to assemble. However, the insert may be secured in the chamber 12 by adhesive, welding, or a ledge or track on the sidewalls 14, if desired.

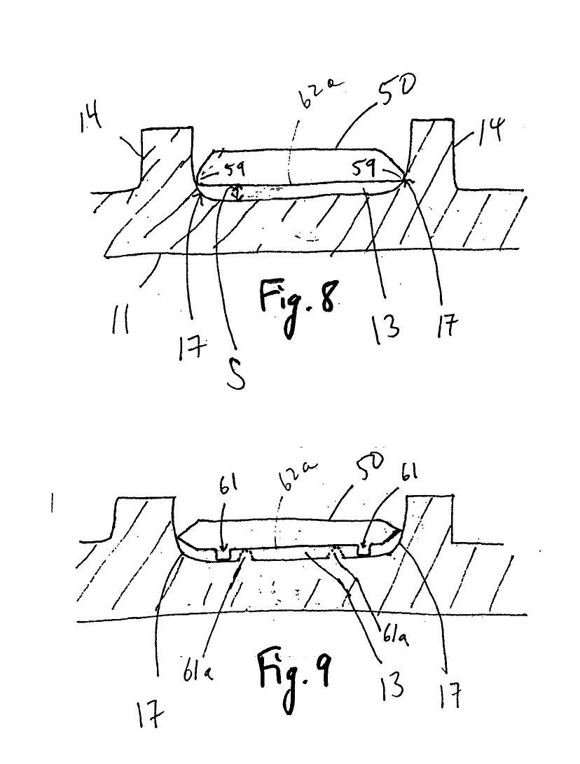

Preferably, the press-fit is provided by protrusions from either the inner surface of the sidewall 14 or the edge of the insert 50. In the preferred embodiment, protrusions are provided on both components. As shown in FIGS. 4 and 5, for example, protrusions or shoulders 64a, 64b and 64c extend from the edge 59 of the insert 50. Protrusions 18a and 18b extend from the inner surfaces of the sidewalls 14 and the bottom wall 16 of the base 11. When the insert 50 is press-fit into the cavity 12, the protrusions 18a contact the shoulders 64a and the protrusions 18b contact the shoulders 64c. The protrusions 64b need not be provided. Portions 66 of the edges of the insert 50 are thereby spaced from the walls 14 of the chamber, forming vents 66, as shown in FIG. 3. The vents 66 allow for the escape of air from the capillary chamber 13 as the chamber fills with the fluid sample, avoiding the formation of air bubbles in the chamber. In addition, the vents space portions of the side edge 59 of the insert sufficiently far from the inner surface of the walls 14 so that any capillary force between those surfaces is less than the capillary force between the planar wall 62 and the bottom wall 16. In particular, the distance between the side edge 59 of the insert 50 and inner surface of the side wall 14 is greater than the distance between the underside 62a of the planar wall 62 and the region 16a of the bottom wall 16. Without such vents, fluid sample could be drawn into and along the small space between the side edge of the insert 50 and the sidewall 14 by capillary force more quickly than the fluid would be drawn into the capillary chamber 13, forming air bubbles in the capillary chamber 13. While the protrusions are not required and the insert 50 could be press-fit in cavity 12 along its entire edge, such a configuration could cause stress which could crack the insert 50. A portion of the edge 59 may also be beveled as shown in FIG. 8, discussed below, to reduce the size of the contacting surfaces, further reducing stress on the insert 50.

The intersection 17 between the sidewalls 14 and the bottom wall 16 is typically curved, since it is difficult to mold sharp edges by molding processes. FIG. 8 is a cross-sectional view of FIG. 1 along line 8-8, showing an edge 59 of the insert 50 supported by the curved intersection 17. The beveled edge 59 of the insert mentioned above is also shown. The widest portion of the insert 50, which engages the intersection 17, is preferably the lowest portion of the side edge, to ensure proper placement of the insert 50 in the chamber 12. The space “S” beneath the planar wall 62 and the bottom wall 16 forming the capillary chamber 13 is also shown. If the cavity 12 is made wider or the insert 50 is made narrower, the insert 50 may rest on the bottom wall 16. In this case, there will still be a sufficient space S between the underside 62a and the bottom wall 16 to form a capillary chamber 13.

For greater control over the distance between the planar wall 62 and the bottom wall 16, longitudinal legs 61 may be provided on the underside 62a of the planar wall 62, as shown in cross-section in FIG. 9. In that case, the widest portion of the beveled edge is preferably not the lowest portion of the edge, so that the legs 61 rest on the major planar wall 16 of the chamber 12. Legs 61a could also be provided protruding from the bottom wall 16, as shown in phantom FIG. 9. Two such legs in the capillary chamber 13 may be sufficient. A leg 61c may also be provided on either the underside 62b or the region 16b of the bottom wall 16, as shown in phantom in FIG. 7A, for greater stability of the insert 50 in the cavity 12. Instead of longitudinal legs, at least three and preferably four or more protrusions may be formed on either the underside 62a or the region 16a. A combination of legs and protrusions may also be provided. For example, the leg 61c may be provided between the underside 62b and the region 16b, while three or more protrusions may be provided between the underside 62a and the region 16a. The legs and/or protrusions are preferably formed during molding of the insert 50 or the base 11. Alternatively, the legs or protrusions may be separately formed and may be attached to the insert or to the base. The insert 50 could also be supported by beads of plastic, silicon or glass between the insert 50 and the bottom wall 16 of the cavity.

Returning to FIGS. 1-5, a cut out guide 17 is preferably provided in the cavity 12 to receive a member 51 in the insert 50, to facilitate proper placement of the insert in the cavity.

The underside surface 62b of the insert 50 outside of the capillary chamber 13 is parallel to the central tapered panel 56, as shown in FIGS. 6 and 7. The distance between the underside 62b of the input portion 52 and the region 16b of the bottom wall 16 below the input portion 52 is large enough so that the capillary force generated between those surfaces is less than the capillary force generated in the capillary chamber 13.

It is preferred that the capillary chamber 13 be completely surrounded by the side walls 14, to slow evaporation of fluid sample from the capillary chamber and to reduce the risk of contamination of the fluid sample.

The space S between the planar wall 62 and the bottom wall 16 defines a region of constant volume for collection of the sample. Since the analysis is conducted on a known volume of sample fluid, quantitative results may be derived through the use of suitable calibration curves, as discussed further, below. The volume selected for the capillary chamber 13 is dependant upon the expected concentrations of the analyte being tested for. As mentioned above, to provide a capillary force in the chamber 13, the distance between the underside 62a of the planar wall 62 and the major bottom wall 16 may be up to about 2 millimeters. The underside 62a and the major bottom wall 16 may be as close as 0.10 micron or less. The length and width of the capillary chamber 13 may be varied as desired to provide a desired volume by suitably varying the dimensions of the insert 50 and the cavity 12. If the analyte is expected to be present in low concentrations, a larger volume is preferred. If the analyte is expected to be present in higher concentrations, a smaller volume may be used. The dimensions of a portable assay device 10 for analyzing food, water and biological fluid samples for common markers and microorganisms, including bacteria, are described below. Larger volumes may be provided, as well. Test systems can be designed which allow for precision testing of very small volumes, in some cases, as small as a few microliters. This facilitates assays of samples having very small volumes, such as a droplet of blood from a pinprick.

Reagents may be provided on the surface of the region 16a of the bottom wall 16 and/or the underside 62a of the planar wall 62, within the capillary chamber 13, to react with and label an analyte in the fluid sample, enabling their detection. The reagents may be any one or more of several known reagents for detecting an analyte in a sample. For example, the reagent may be a detection protein, such as an antibody or antigen, which is specific to the analyte. The detection protein may be bound to either or both of the walls of the capillary chamber 13 and project into the spaces. An appropriate analyte in the fluid solution that binds to the detection protein will thereby be immobilized by the detection protein. A labeled reagent, also specific to the same analyte, is also provided in the capillary chamber 13 or mixed with the fluid sample prior to its being drawn into the capillary chamber 13, as discussed further below. The labeled reagent binds to the analyte in the fluid sample, before or after the analyte binds to the detection protein. The labeled reagent may then be detected by several techniques, as is known in the art. Suitable labels include fluorescent labels, chemical labels, colorimetric labels, radioactive labels and heavy metals such as gold. The detection protein may also be an analyte-binding protein that is linked to an enzyme that produces a colored reaction product upon incubation with an appropriate substrate, as in ELISA-type assays.

Reagents may be coated, printed or otherwise bound to one or both of the underside 62a of the planar wall 62 and the region 16a of the bottom wall 16 in the capillary chamber 13, using one of several techniques well known in the art. Numerous techniques for applying immunoassays are known in the art and are described, for example, in “Principles and Practice of Immunology” (1997), C. P. Price and D. J. Newman, eds. (Stockton Press) which is incorporated by reference herein, in its entirety.

The analyte-specific reagents may be printed on the interior surface of the plate using a protein printer. Suitable protein printing devices are well known in the marketplace. A contact printer, such as The Virtex Chipwriter™ from Virtex Vision Corporation, Waterloo, Ontario, Canada, for example, is preferred. Other types of printers include ink jet, spray, piezo-electric and bubble jet protein printers. The reagents may be applied in the form of a strip or lane. Test spots may also be provided. Several different analyte-specific detection molecules may be provided to define different lanes or spots for detecting different analytes simultaneously in the same fluid sample. Background and calibration lanes or spots can also be provided. While the reagents may be printed on either or both walls, it is generally easier to print the reagents on the underside 62a, because the insert 50 is smaller than the base 11. Alternatively, reagents in liquid form may be placed onto either or both walls and allowed to dry. For example, luciferin/luciferase reagents for detecting adenosine triphosphate (“ATP”), are typically applied in this manner. After placement of the reagents, the insert 50 is press-fit placed into the chamber 12.

The binding of protein test and calibration spots or lanes may be improved by coating the surface where the spots or lanes are to be applied with a protein immobilizer. One suitable protein immobilizer is Fast Red B salt (4-nitro-2-methoxy-aniline; C.I. Number 37125). Fast Red B base has a molecular formula of C₇H₈N₃O₂, and a molecular weight of 168. Fast Red B salt has the following structure:

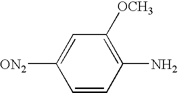

The part of the assay device 10 to receive the protein detection reagents, preferably the insert 50, is incubated in a solution of 1 mM Fast Red salt in phosphate buffered saline (“PBS”) pH 7.4, at 30° C. for 60 minutes, followed by washing under ultra-pure, deionized water, such as double distilled water, three times, for 5 minutes each. Double distilled water may be provided by a Milli-Q filtration unit from the Milllipore Corporation, Burlington, Mass., U.S.A., for example. If the assay devices 10 are to be stored prior to use, it is recommended that they be stored at 4 degrees Celsius and be protected from light. The Fast Red B salt has been found to improve wettability, as well.

In order to reduce the background noise and therefore increase the sensitivity of the assay, a mask (not shown) may be provided adjacent to the reading portion 54 of the insert 52 or adjacent to the base 11. The mask is an opaque material with openings corresponding to the lanes or spots of reagents. An example of a mask is shown in WO 00/78917 A1, published on Dec. 28, 2000, (based on PCT/US00/13056, filed on May 12, 2000) and WO 00/29847, published on May 25, 2000, (based on PCT/CA99/01079, filed on Nov. 12, 1999), which are assigned to the assignee of the present invention and incorporated by reference herein, in their entireties.

The surface of the insert 50 and the base 11 which come into contact with the fluid sample are preferably smooth. A smoothness having optical quality is most preferred.

Depending on the reagent and the analyte, it may be advantageous to support the reagents in a carrier positioned in the chamber 12 prior to placement of the insert 50. For example, the reagents may be bound to a thin layer of a dry matrix material, such as cellulose, or to a thin layer of gel, such as agar. The agar provides a growth medium for the analyte, as well.

A separating medium may be provided in the input portion 52 of the insert 50, to separate the fluid component of the sample from unwanted components of the sample greater than a predetermined size. As mentioned above, in testing blood, for example, erythrocytes, leukocytes and platelets are typically separated from the plasma prior to testing. Test samples from water supplies and food typically contain solid matter which needs to be separated from the liquid prior to testing. The separating medium is preferably provided in the passage or passages 60.

A preferred separating medium is a collection of microspheres or beads which, when exposed to fluid, move and transiently abut each other. The interstitial spaces or pores between the microspheres are also, therefore, transient. It is believed that the fluid is drawn through the interstitial spaces between the microspheres by capillary force. Such a separating medium is therefore referred to as dynamic capillary filter, as discussed in WO 00/78917 A1, published on Dec. 28, 2000 (based on PCT/US00/13056, filed on May 12, 2000), and WO 00/29847, published on May 25, 2000 (based on PCT/CA99/01079, filed on Nov. 12, 1999, assigned to the assignee of the present invention and incorporated by reference herein, in their entireties. See also U.S. Ser. No. 09/335,732, filed on Jun. 18, 1999, which is also assigned to the assignee of the present invention and incorporated by reference herein, in its entirety.

FIG. 7 shows a plurality of microspheres 68 in the passages 60. The microspheres 68 are supported within the passages 60 by a porous material 70 with pores smaller than the diameter of the microspheres. Sample fluid thereby passes through the porous material 70 while the microspheres are supported by the porous material. The porous material may be a nylon or polyester mesh 70, for example. The porous material 70 is preferably attached to the bottom surface of the wall 62, covering the exit 60b of the passages 60. Alternatively, the porous material 70 may be placed in the passages 60.

The microspheres 68 may have a uniform spherical shape. Preferably, the microspheres have a diameter of between about 0.5 up to about 100 microns, for filtering biological fluid samples, or fluid samples, derived from food or water supplies. More preferably, for these applications, the microspheres have a diameter in a range between about 5 to about 15 microns. Microspheres with non-uniform shapes may be used, as well.

Since the analyte must be able to pass through the interstitial spaces of the microspheres, the size of the microspheres for a particular application depends on the size of the analyte and the size of the unwanted material. It is preferred to use microspheres as large as possible but still able to remove the unwanted material, because smaller microspheres slow the advance of the fluid sample into the capillary chamber 13. The size of the space formed between the microspheres 68 is a function of the radius of the curvature of the microspheres. The radius of curvature is, for the purposes of the present invention, the same as the diameter of the microspheres. It is known that the ratio of the microsphere diameter to the pore diameter is approximately 1 to 0.4. To separate plasma from whole blood, for example, a pore size of 4 microns is considered optimal. Therefore, the preferred microsphere diameter is 10 microns. This permits an easy fluid flow (and therefore faster fluid flow) while still preventing cells from passing through the pores. When the detection of bacterial contaminants of a food or water sample is desired, the optimal microsphere diameter is 15 microns, due to the larger size of the typical unwanted materials in the sample. This provides pore sizes of 6 microns.

The size of the microspheres 68 used to separate the fluid component can also be varied based on the viscosity of the sample. Larger microspheres may be used for more viscous samples for faster fluid flow between the beads, as long as the resulting pore sizes are small enough to capture the unwanted material and large enough to allow the analyte to pass through.

The pore size of the porous material 70 should be sufficiently less than the size of the microspheres 68, to prevent passage of the microspheres through the porous material. For example, where the microspheres have a diameter of 15 microns, the porous material preferably has a diameter of 11 microns. Where the microspheres have a diameter of 10 microns, the porous material 70 preferably has a pore size of about 1-2 microns.

In a preferred embodiment, latex microsphere beads, such as those available from Bang's Laboratories, Inc., Fishers, Ind., U.S.A., are used. The beads are supplied in a liquid suspension. Other types of particles could also be used, including glass particles, silica particles and sand particles.

A suitable nylon mesh 70 with a pore size of 10 microns is available from the Millipore Corporation, Burlington, Mass., U.S.A., for example, under the catalog number NY11, comprising a weave of 6, 6 polyamide with square pores. A suitable polyester mesh 70 with a pore size of 7 microns is available from Saati Tech, Inc., Somers, N.Y., U.S.A., for example. Polycarbonate films with pore sizes of either 5 or 10 microns are also available from the Millipore Corporation under the trade name ISOPORE. The polycarbonate films provided by Millipore are coated with polyvinylpyrrolidone (PVP) as a wetting agent. A polysulfone membrane 70 with a pore size of 1.2 microns, which is preferred for use with assaying blood (with microspheres 68 having a diameter of 10 microns), is available from Osmonics, Inc., Westbury, Mass., U.S.A. Filter papers may also be used.

Instead of the passages 60 extending completely through the insert 50, a thin layer of material can close the exit 60b of the passages 60, to support the microspheres 68. Small holes or slits of appropriate size can be made through the thin layer to allow for fluid flow while supporting the microspheres. The holes or slits may be formed by laser, for example.

The porous material 70 may be attached to the underside 62 of the planar wall 62 by a solvent or by sonic welding, for example. Solvents which may be used include methyl ethyl ketone, acetone, toluene and benzene. Since a solvent dissolves the plastic of the insert 50, extra plastic material, in the form of a protruding ring 71, is preferably provided at the site where the porous material 70 is to be attached, as shown in FIG. 6. To apply the porous material 70 to the insert 50, the material 70 is placed over the protruding ring 71. The material 70 is swabbed with the solvent, which soaks through the material. The solvent melts the protruding ring 71. The melted plastic hardens, bonding to the porous material 70. It is important that excess amounts of solvent not be used, so that the melted plastic does not fill and close the pores of the material 70.

After the porous material 70 is attached to the insert 50, the microspheres 68 are inserted into the passages 60. The microsphere beads provided by Bang's Laboratories, Inc. are suspended in a liquid solution comprising 0.02% sodium azide preservative in water. The suspension comprises 10% microspheres (weight per volume) in the liquid solution. The passages 60 are filled with the suspension, through a pipette or other small tube. The liquid drains from the microspheres 68, through the porous material 70. The microspheres 68 are then preferably dried, by allowing the liquid to evaporate overnight or by placing the insert 50 into a chamber with a dry atmosphere, for example. While wet microspheres may be used, the fluid on the microspheres could change the concentration of the analyte in small fluid samples.

When a fluid sample is applied to the microspheres 68, the microspheres again become suspended in the fluid. The suspension of the beads in the upper portion of the passage may be less dense than the suspension in the lower portion of the passage. A gradient filtration effect may thereby be created, wherein larger particles are caught in the upper portion of the passage while smaller particles are caught in the lower part of the passage.

Reagents may optionally be provided among the microspheres 68. For example, the microspheres could be impregnated with or bound to the labeled reagent, (such as a fluorescently labeled antibody), for example, by adsorption or coupling. As the fluid passes through the capillary channels formed by the microspheres, the analyte will mobilize the labeled reagent contained on the microspheres. The labeled analyte then reacts with the detection reagents (such as another antibody) which may be bound to a wall surface of the capillary chamber 13. Anticoagulants may also be provided on the microspheres to prevent blood clotting prior to a sufficient amount of a blood sample passing through the microspheres, into the capillary chamber 13.

The microspheres 68 may also be treated with a protein blocking agent, such as bovine serum albumin (“BSA”) or a polysaccharide, such as hydroxypropylmethyl cellulose (“HPMC”) to lessen the binding of analyte to the latex microspheres 68, described above. More analyte is therefore drawn into the capillary chamber 13 and is available for analysis. The signal intensity of the labeled analyte, such as a fluorescently labeled analyte, is thereby increased. To coat the microspheres 68, 200 microliters of 10% weight by volume (“w/v”) microsphere beads from Bang's Laboratories, described above, were diluted with 1 mL phosphate buffered saline (“PBS”) pH 7.4, and mixed. This solution was centrifuged for 10 minutes at 14,000 rpm in a bench-top microfuge. The supernatant was removed. The remaining pellet of beads was resuspended in 1 ml PBS ph 7.4 and the washing procedure was repeated two more times. After the third centrifuge cycle, the pellet was resuspended in 1 ml of PBS ph 7.4 containing 10 milligrams per milliliter of BSA. This suspension was incubated overnight at room temperature with end-over-end mixing. Following incubation, the treated microspheres were washed three times in PBS pH 7.4, as described above. The final bead pellet was resuspended in 200 microliters PBS storage buffer containing: 10 mg/mL BSA, 0.1% sodium azide and 5% glycerol. The suspension was stored at 4 degrees Celsius until used. BSA is available from Sigma-Aldrich Corporation, Milwaukee, Wis., U.S.A., for example.

In another process, the washed pellet of beads was resuspended in 1 mL of 1% weight per volume (“w/v”) HPMC and incubated for 1 hour at room temperature with end-over-end mixing. Following incubation, the treated microspheres were washed three times in PBS pH 7.4, as described above. The final bead pellet was resuspended in 200 microliters of ultra-pure, deionized water, such as double distilled water from a Milli-Q filtration unit, available from the Millipore Corporation. Preferably, the HPMC is high molecular weight HPMC, with an average molecular weight of 86,000, for example.

More than one size of microspheres 68 may be present. Smaller microspheres could nestle in the interstitial spaces formed by the larger microspheres. The smaller microspheres could carry the labeled reagent for binding to the analyte as it passes through the microspheres.

Providing the separation medium within the assay device 10 simplifies the testing process by eliminating the need for a separate separation step prior to application of the sample to the assay device 10. This enables the assay 10 to be used at the point of patient care, for example, by the patient, at the patient's bedside or in a doctor's office. In food and environmental testing, the assay device can also be used in the field, at the source of the sample. In addition, the microspheres 68 of the present invention provide improved fluid flow without restriction by the fiber in the chromatographic paper or other fibrous materials used in the prior art to wick the fluid component of a biologic sample away from the cellular component.

While incorporating the separation medium in the assay device 10 is one advantage of the present invention, there may be times when a separate filtration step is preferred. Separation may be provided by centrifugation, for example. It may also be advantageous to concentrate the analyte by centrifugation. Centrifugation has been used for the concentration of bacteria, for example. Immunomagnetic bead concentration and separation techniques can be used to concentrate bacteria and to separate the bacteria from unwanted components of the fluid sample. Certain water samples may not need filtration, either. Whether filtration is required or not, providing the microspheres 68 in the passages 60 is still preferred, because it has been found that the microspheres improve the fluid flow through the passages 60.

If the microspheres 68 and the porous material 70 are not desired, however, they need not be provided. But it has been found that if the bacteria are separated by immunomagnetic bead separation, bacteria-magnetic bead complexes may collect at the protruding ring 71. Therefore, when the porous material 70 is not provided, it is preferred that the ring 71 be removed. The ring may be removed by placing the porous material 70 on the ring and applying a solvent, such as acetone, onto the porous material. When the acetone dries, the porous material is removed. The ring 71 is thereby partially removed and flattened. Alternatively, the insert 50 may be molded without the ring.

Preferably, an upstanding wall 72 is provided around the input portion 52 to prevent fluid sample from passing around the edge 59 of the insert 50 without passing through the passages 60 and the microspheres 68 (See FIGS. 2 and 5). The wall 72 also protects the fluid sample within the input portion from being smeared or wicked away by a foreign object. A portion 74 of the wall is preferably adjacent to the passages 60 to provide a guide for a pipette, syringe or other such application device, assisting in the placement of the fluid sample in or close to the passages 60. Portions 76 of the wall 74 may be curved to follow the curve of the passages 60, as shown in FIG. 2. The portion 74 also helps prevent the movement of fluid sample onto the reading portion 54.

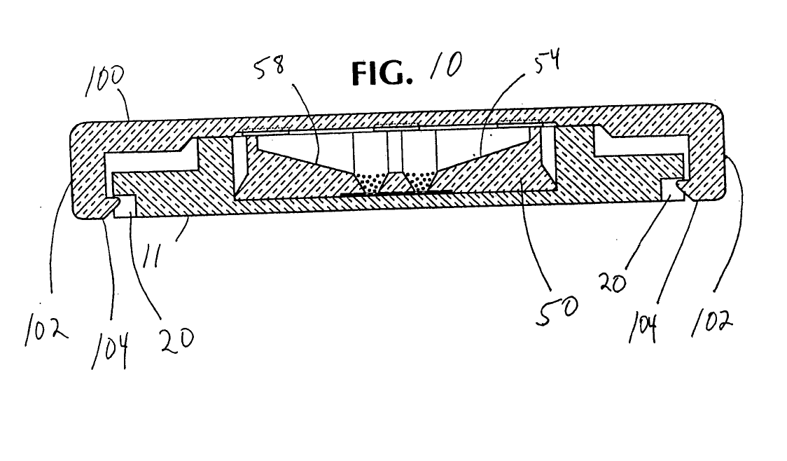

The lid 100 is preferably provided to selectively cover a portion of the insert 50, as shown in FIG. 1. The lid 100 helps prevent contamination of the sample after application onto the assay device 10. The lid also contains excess sample which cannot be drawn into the capillary chamber 13. Humidity in a region proximate the fluid sample is also maintained by the lid 100, slowing the evaporation of the fluid so that the fluid sample does not dry prior to being drawn through the passages 60, into the capillary chamber 13. The lid also helps prevent evaporation of fluid from the capillary chamber 13. FIG. 10 is a cross-sectional view of FIG. 7 along line 10-10. FIG. 10 shows the lid 100 with sidewalls 102 and flanges 104 that snap over the edge portions 20 of the base 10. The edge portions 20 can be in the form of grooves with a defined length to limit the range of movement of the lid. The lid 100 can slide back and forth along the region 20.

Prior to use, the lid 100 preferably covers the reading portion 54 of the insert 50, exposing the input portion 52. After application of the sample to the input portion 52, the lid is preferably slid over the input portion 52. The reading portion 54 is then exposed for viewing. Protrusions 24 may be provided on the rear wall 14a of the base 11, adjacent to the input portion 52, for engaging a rear edge of the lid, locking the lid in place by a snap fit. (See FIGS. 1 and 4, for example).

In one preferred configuration, the assay device is portable. The base 11 has a length of 76 millimeters (“mm”) and a width of 25.4 mm. The length of the interior of the chamber 12 is 20 mm. The width of the interior of the chamber is 14 mm. The first protrusions 18a protrude 50 microns into the interior of the chamber 12. The second protrusions 18b protrude 190 microns into the interior of the chamber. The insert 50 has a length of 20 mm and a width of 14.0 mm, including the protrusions 64a and 64b. The protrusions 64a and 64b extend 0.5 mm from the side edge of the insert 50. The second protrusions 64c extend 360 microns from the side edge. The vents 66 have a width of about 550 microns. The input portion 52 has a length of 11 mm. The reading portion 54 has a length of 8.5 mm and a width of 13.0 mm. As discussed above, the distance between the surface 62a of the planar wall 62 and the major bottom wall 16 of the base is preferably from about 10 microns to about 120 microns. The preferred volume therefore ranges from about 1.1 microliters to about 13 microliters. The angle of the central tapered panel is 8° from horizontal in FIGS. 6 and 7. Each passage 60 has an entrance 60a with a diameter of 1.60 mm and an exit 60b with a diameter of 0.80 mm. The protruding ring 71 has a diameter of 2.10 mm and extends 0.08 mm from the bottom surface of the insert 50. The porous material 70 has a length of 6.20 mm and a width of 3.40 mm. A nylon mesh 70, for example, has a thickness of 0.065 mm.

The lid 100 has a length of 28.4 mm and a width of 14.2 mm.

While in the preferred embodiment the region 62a of the insert and the region 16a bottom wall 16 of the base 11 are planar, those surfaces may be curved as well. It is preferred that those surfaces be parallel when light intensity or color are to be detected. Otherwise, those walls need not be parallel.

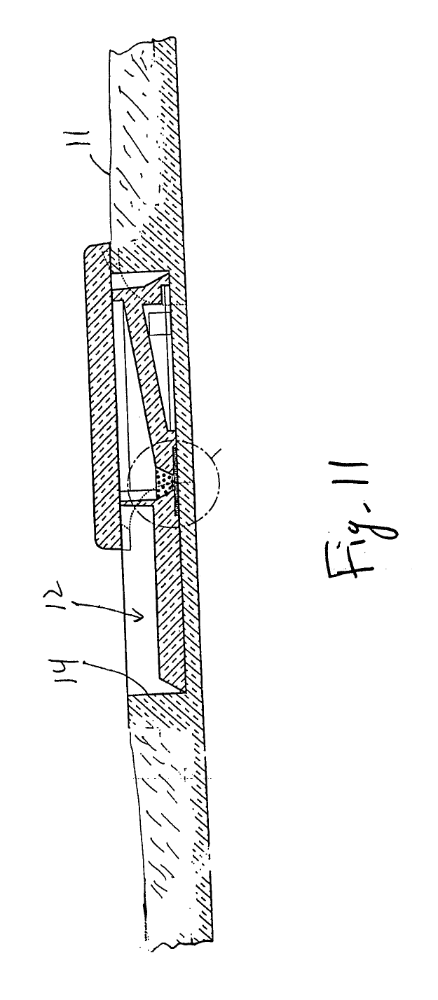

The cavity 12 may be recessed in the base 11, as shown in FIG. 11. The base 11 is much thicker than in FIGS. 1-9, to accommodate the cavity 12. The sidewalls 14 of the cavity 12 are wall surfaces within the base 11.

All types of materials which may bind reagents or may be surface treated to bind reagents, may be used in the present invention. However, either the insert 50 (or at least the reading portion 54) or the base (or at least the portion of the bottom wall 16 below the reading portion 54), should be transparent. For example, glass and plastic may be used. Metal may be used for certain components. Preferred materials for the assay device 10 are polystyrene, polycarbonate, polypropylene and poly(methylmethacrylate). Poly (methylmethacrylate) is preferred for bioluminescent assays such as luciferin/luciferase assays for detecting adenosine triphosphate (“ATP”) because poly(methylmethacrylate) has relatively low absorption at the bioluminescent emission bandwidth of the reaction. It is also noted that if the assay device is made of polystyrene, green fluorescent dye may not be used as a label.

Polystyrene, polycarbonate and polypropylene, the preferred materials for the assay device 10, are hydrophobic. This could interfere with the flow of the fluid sample into the capillary chamber 13. Therefore, the surfaces of the assay device 10 not coated with reagents is preferably treated with a wetting agent. Since it is preferred to apply the reagents to the underside 62a of the insert 50, the bottom wall 16a of the cavity is preferably treated with the wetting agent. Polysaccharides (cellulose derivatives) are preferred. Hydroxypropylmethyl cellulose (“HPMC”) is most preferred. HPMC has been found to be particularly useful if the base 11 is made of polystyrene. A wetting agent is not needed if the base is acrylic, but it may be used, if desired.

It has also been found that polysaccharides such as HPMC reduce the non-specific binding of proteins, such as labeled antibodies, to the surfaces of the planar wall 62 and the bottom wall 16. The background contrast with the labeled and bound analyte is increased and is more uniform. In addition, removal of the analyte not bound to a test spot by flushing or drawing the fluid sample out of the capillary chamber, which is advantageous in certain assays to improve background contrast, is facilitated. High molecular weight HPMC has been found to give the best protein blocking and wetting effects. An average molecular weight of at least about 86,000 is preferred.

High molecular weight HPMC may be applied to the base 11 by filling the cavity 12 with an aqueous solution (1% weight by weight) of the HPMC. After about one hour, the base 11 is washed with water and allowed to dry. The entire base 11 may also be immersed in the aqueous solution for one hour, washed and allowed to dry. HPMC may be obtained from Sigma-Aldrich Corporation, Milwaukee, Wis., U.S.A., for example. The input portion 52 of the insert 50, including the passages 60 may also be coated with HPMC to improve fluid flow. A low molecular weight HPMC may also be used as long as the HPMC is applied for a larger period of time. For example, if the HPMC has an average molecular weight of about 26,000, it is preferably applied for about 4 hours. Other protein blockers, such as BSA, discussed above, and skim milk, may also be used.

Since it may be difficult to differentiate between an assay device 10 with a negative result (no analyte present), an assay device whose reagents have denatured, or an assay device that has not actually been used, a control spot is preferably provided in the capillary chamber 13. The control spot may be a substance which will bind to the same labeled reagent as the analyte. The labeled reagent may be an antibody with a fluorescent label, for example. When a fluid sample enters the capillary chamber 13, the labeled reagent will bind to the control spot, as well as to the analyte in the fluid sample, if present. The labeled reagent may then be detected on the control spot, even if no analyte is in the sample, indicating that the assay device 10 has been used and the reagent, or at least that the labeled reagent, is effective. Preferably, the control spot is placed in a predetermined location, such as in the center of capillary chamber 13, so that it may be easily located by a human or machine reader.

Use of the assay device 10 will now be described. As mentioned above, some or all of the reagents may be mixed with the fluid sample prior to application to the assay device 10. For example, detection antibodies may be printed on a surface of the capillary chamber 13 and labeled antibodies may be mixed with the fluid sample prior to application to the assay device 10. The labeled antibodies may be mixed with the fluid sample after removal from a culture medium, for example. Labeled antibodies may also be provided among the microspheres. In certain assays, such as bacterial detection assays, only a labeled antibody is used.

To use the assay device 10 in accordance with the present invention, a fluid sample is placed in the input portion 52. The fluid sample may be drawn directly from a source, such as from a water supply or a bodily fluid and may be applied by known techniques, such as a pipette. A syringe may also be used. A drop of blood could be applied directly from a pinprick to the input portion 52. The fluid sample may also be drawn from a culture medium. The fluid sample may be concentrated prior to application to the assay device 10 by contacting the sample with a superabsorbent polymer, such as those containing acrylamide and/or dextran, which are capable of absorbing large amounts of water and/or small ionic species. The superabsorbent polymer may be held in a syringe, or other suitable container, and the sample mixed with the polymer while in the syringe. The polymer gel in the syringe may also include a reagent to be exposed to the sample during the concentration step. A labeled antibody, such as a fluorescently labeled antibody, may be provided, for example. After the concentration step is completed, the concentrated sample is expressed from the syringe and applied to the input portion 52 of the assay device 10.

Preferably, the fluid sample is placed over the passages 60 but since the panels 54, 56, 58 are tapered towards the passages 60, sample placed anywhere in the input portion 52 will be drawn to the passages by gravity. The fluid sample may be about 5 microliters to about 65 microliters, for example, depending on the size of the capillary chamber 13. Preferably, the amount of the fluid sample applied is greater than the volume of the capillary chamber 13 by a sufficient amount so that after filtration, there is still excess fluid sample in the input portion 52 and the passages 60. This helps slow the evaporation of the fluid sample from the capillary chamber 13. The lid 100 is then preferably slid over the input portion 52 and locked in place, exposing the reading portion 54 and securely covering the input portion 52. The fluid sample is drawn through the passages 60 and through the microspheres 68, if present, by capillary force and gravity to remove materials over a predetermined size. The filtered fluid sample exits the passages 60 at the entrance 15 of the capillary chamber 13. The filtered fluid sample is then drawn into the capillary chamber 13. In the capillary chamber, the analyte in the fluid reacts with the reagents in the chamber.

After the analyte reacts with the reagents, a measurable reaction product exists in the capillary chamber 13. The base 11 and the insert 50 of the assay device 10 are preferably colorless or transparent so that calorimetric, fluorescent, chemiluminescent, bioluminescent or other reaction products can be detected by techniques well known in the art. For example, the reaction products maybe read visually, under a microscope. A photoconductive detection device, such as a photodiode, a photomultiplier or a CCD, may also be used. A detecting device, such as a spectrophotometer, a luminometer, a fluorometer or another appropriate detector coupled to a reader may also be used, as is known in the art. The intensity of the reaction product may be measured to determine the amount of analyte present in the sample by comparison to calibration curves, for example, as discussed above.

The assay device 10 may be designed to be read by a portable spectrophotometer which reads, for example, the change in color after the analyte has reacted with the labeled antibody. A Genepix Spectrophotometer, available from Axon Instruments, Inc., Foster City, Calif., U.S.A., may be used, for example. Once the spectrometer, or other such detector, has performed the necessary data calculations, the results are transmissible by digital transmission over the telephone lines, by cell phone, or other computer network system.

The detector may be moved with respect to the reading portion 54 or the reading portion may be moved with respect to the detector, automatically or manually.

As mentioned above, quantitative determinations may be obtained with the assay device of the present invention through calibration curves. A plurality of calibration spots or lanes, each comprising a binding reagent of a different, known quantity, may be provided in the capillary chamber 13. A sufficient number of calibration sites should be provided to enable accurate interpolation between the sites to generate a calibration curve on a graph. The intensity (light intensity or color intensity) of the analytes binding to the test spots may be compared to the intensity of the calibration sites, to determine the concentration or quantity of the analyte in the fluid sample. The comparison may be conducted visually under a microscope, by directly comparing the test and calibration spots. More accurate determinations may be made by a comparison of a quantitative measure of the intensity obtained by spectrophotometer, for example, with the calibration curve. The comparison may be conducted by a computer. To obtain truly quantitative data, incubation steps for the purpose of culturing more analyte are often emitted, so that a measure of the actual amount of analyte in the original sample may be obtained.

Fluorescent emissions from a fluorescently labeled analyte may be detected using a fluorometer. Information about the distribution of fluorescent emissions, including location and intensity, can be obtained by acquiring an image using a CCD camera and commercially available software, such as microassay analysis software, such as GenePix Pro™ from Axon Instruments, Inc. Image-Pro™ 4.1, available from Media Cybernetics, Silver Spring, Md., U.S.A., is useful for counting fluorescently labeled bacteria.

In another alternative, changes occurring during an antibody/analyte reaction may be detected or measured by changes in radio frequency if a radio frequency sensor (not shown) is incorporated into one of the plates of the assay device 10.

The assay device 10 is preferably discarded after use, following appropriate, standard hazardous waste guidelines.

As mentioned above, unbound, labeled analyte in the fluid sample in the capillary chamber 13 may interfere with the optical analysis of bound analyte. For example, when measuring the intensity of the analyte, such as soluble protein, bound to a spot of reagent to determine the concentration of the analyte in the fluid sample, unbound, labeled analyte in the fluid sample may decrease the contrast of the background of the spots. The sensitivity of the measurement is therefore decreased. It may therefore be advantageous to remove the fluid sample from the capillary chamber 13 prior to analyzing the test results. The fluid sample may be wicked from the capillary chamber 13 by an absorbent material such as cellulose fibers, for example. Preferably, the chamber is refilled with water after the fluid sample is removed and prior to optically analyzing the reaction products.

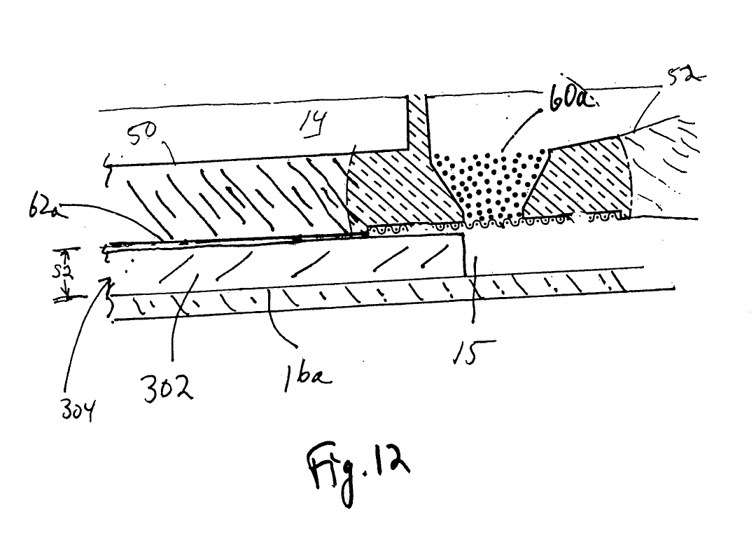

The spaces between the planar wall 62 and the bottom wall 16 need not define a capillary chamber. In another embodiment of the invention, shown in partial cross-section in FIG. 12, the distance between the underside 62a of the planar wall 62 and the major planar wall 16 is greater than about 2.5 mm. The space “S2” between these surfaces does not, therefore, define a capillary chamber. The remainder of the assay device is the same as in the first embodiment and common elements are commonly identified. A thick layer of a carrier material 302 is provided on the region 16a of the bottom wall 16. Reagents may be provided in the carrier material 300. Fluid sample applied to the input portion 52 and passing through the passages 60 to the entrance 15 diffuse into the carrier material 302 in the chamber 304. The insert 50 is provided in the chamber, over the carrier 302. In the assay device 10 described above, the carrier layer 304 may be any desired thickness as long as the insert 50 is not above the height of the sidewalls 14 of the cavity 12. The height of the side walls may be adjusted as necessary.

The carrier material 302 may be a gel, such as biopolymer hydrogel, such as agar, for example. Agar may act as a culturing medium, in situations where incubation of the analytes would be advantageous. In addition, the presence of the gel slows the movement of the analyte in the sample, in situations where more time may be required for the reaction to be completed. The use of a thick layer of gel also enables the progress of the reaction to be monitored. Agar may be purchased in granular form from Becton, Dickinson & Co., Sparks, Md., U.S.A., for example. The granulated agar may be mixed with water or a buffer solution to form a matrix gel.

The carrier material 300 may also be a thick layer of a dry matrix material such as cellulose.

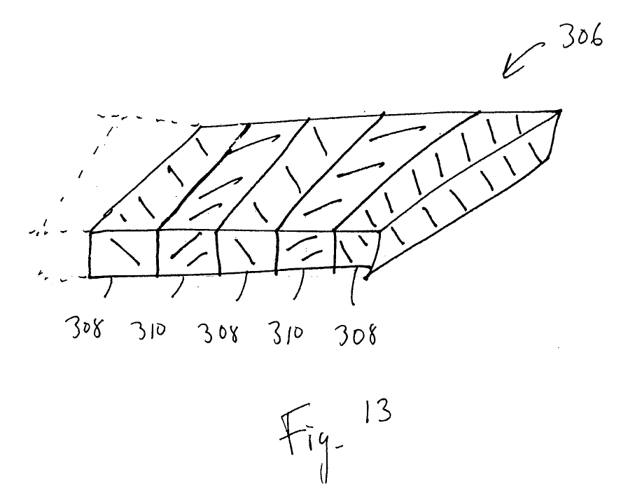

In another non-capillary embodiment, a multi-layer carrier 306 is provided in the space between the planar wall 62 and the bottom wall 16. A perspective view of a multi-layer carrier is shown in FIG. 13. The carrier 306 may comprise a dry matrix layer 308 of cellulose, for example, followed by a gel layer 310, such as agar. Additional dry and gel layers may be provided, as needed, as shown in FIG. 13. Different reagents may be provided in each layer. As the fluid sample diffuses through each layer of the carrier, the reaction products produced in each layer encounter different reagents in a subsequent layer. In this way, the progress of complex reactions may be controlled and monitored. Since a fluid sample diffuses through a dry matrix faster than it diffuses through a gel, reagents requiring more time to react with the analyte or the current reaction product may be provided in the gel while reagents requiring less time to react may be provided in the dry matrix. The width of the layers may also be adjusted to control the diffusion time. The layers may be vertically stacked, as well. The use of multi-layer carriers could be particularly useful in assays for fungi and mold, and other microorganisms which require multiple reactions and incubation time in a gel to progress. Certain immunodiagnostic assays, as well as the identification of chemical species, could also be facilitated by multi-layer carriers.

As mentioned above, the assay device 10 of the present invention may be used with bioluminescent and chemiluminescent assays. As is known in the art, chemiluminescence is the production of light by a chemical reaction and bioluminescent is a type of chemiluminescence wherein the production of light is by a biochemical reaction.

The detection of adenosine triphosphate (“ATP”) as an indicator of the presence of living cells in a sample using a luciferin/luciferase reaction is a well-known example of a bioluminescent assay wherein a biochemical reaction results in the production of light. The bioluminescence may be detected with a photomultiplier tube (“PMT”), for example. The intensity of the bioluminescence and the electrical current generated by the PMT are proportional to the amount of ATP present in the sample. ATP bioluminescent assays can be used to determine the presence of biological contamination in areas where food products, such as meat, poultry, milk, wine and beer, for example, are prepared.

Luciferase may also be used to detect and quantify bacterial levels in a sample based on its reaction with flavin mononucleotide (“FMN”), which also produces light. See, for example, Picciolo, G. L., et al., “Applications of Luminescent Systems to Infectious Disease Methodology,” Goddard Space Center Publication X-726-76-212, September 1976, pp. 60-68. Light emission is proportional to the amount of FMN in the sample. FMN concentration can be related to bacterial levels or used to evaluate abnormal flavin concentrations, which may relate to metabolic disorders.

Bacteria can also be detected in fluid samples by the reaction of iron porphyrines, such as peroxidase, cytochrome and catalase, which are present in biological cells, with luminol (5-amino 2,3-dihydro-1,4-pthalazine-dione) to produce visible light. See U.S. Pat. No. 4,234,681; Picciolo, G. L., “Applications of Luminescent Systems to Infectious Disease Methodology,” pp. 69-73, cited above. The reaction of luminol with hydrogen peroxide in an aqueous alkaline solution in the presence of an oxidizing activating agent such as ferricynamide, hypochlorite or a related transition metal such as iron or copper, produces chemiluminescence.

The assay device 10 of the present invention may be used to conduct bioluminescent and chemiluminescent assays by placing a desired amount of a reagent, such as luciferin/luciferase, into the capillary chamber. To detect ATP, 10 microliters of luciferin/luciferase reagent may be placed into the cavity 12 of the assay device 10 of the dimensions described, above. Preferably, the cavity has been treated with HPMC or another polysaccharide wetting agent. The reagent is allowed to dry and then the insert 50 is placed in the chamber 12. The luciferin/luciferase reagents may also be provided in the chamber in lyophilized form. The reagent includes a lytic agent, such as ethylenediaminetetraacetic acid (EDTA) to dissolve bacterial cell walls, causing release of the ATP, as is known in the art.

A sample is collected from a surface to be tested in a known manner, such as by use of a wet swab, and applied to the input portion 52 of the assay device 10. Since dirt and other such materials do not typically interfere with analysis of luminescent reaction, it is not necessary to provide microspheres 68 in the passages 60, for filtering the fluid sample. They may be provided, if desired. ATP in the sample reacts with the luciferin/luciferase reagents and if ATP is present, light is emitted.

As was also discussed above, for bioluminescent tests, the assay device 10 is preferably made of poly(methylmethacrylate), which has a low optical absorbance at the bioluminescent emission wavelength of 562 nanometers for the luciferin/luciferase reaction for ATP. Polystyrene and polycarbonate may also be used, but are less preferred.

In accordance with another aspect of the present invention, the number of photons of light generated by the reaction which can be detected by the PMT or other such detector is enhanced by providing a reflective surface beneath the base, on an opposite side of the reading portion 54 of the assay device 10 as the detector.

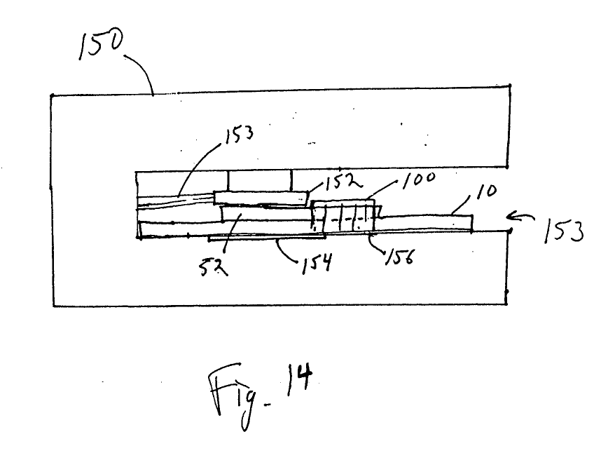

FIG. 14 is a schematic representation of a luminometer 150 including a PMT 152 or other suitable detector mounted within a chamber 153 for receiving an assay device 10. Wires 153 are shown, extending from the PMT for connection to electronics in the luminometer for analyzing the output of the PMT. The assay device 10 is supported by a wall 156 of the chamber. A reflective layer 154 is provided on the wall 156, opposite the PMT 152. When the assay device 10 is inserted into the chamber, the reading portion 52 of the assay device 10 is positioned adjacent to the photodetective surface of the PMT 152 and over the reflective surface 154. Photons emitted in a direction opposite the PMT 152 are reflected by the reflecting surface, toward the PMT, increasing the total number of photons detected by the PMT. The luminometer may include an R928 or a 931B PMT, including accompanying electronics, available from Hamamatsu Corp, Bridgewater, Conn., U.S.A, for example.

Preferably, the photodetective surface of the PMT has a shape matching the shape of the reading portion 52, which in this case is rectangular. The distance between the reading portion 52 and the photodetective surface is as small as possible. Since the intensity of the light decreases with the square of the distance from the source, minimizing the distance between the fluid sample in the capillary chamber 13 and the photodetective surface decreases a significant source of signal loss.

Use of a reflective surface could improve the sensitivity of measurements in assay devices of varying designs. However, the sensitivity of measurements is particularly improved by providing a reflective surface in conjunction with the assay device 10 of the present invention. Since the surface area of the fluid sample in the capillary chamber 13 is large in relation to the volume of the fluid sample, relatively few photons emitted towards the detector are absorbed by the solution. In prior art configurations, in contrast, where the surface area is smaller in relation to the volume, many photons are reabsorbed. Such photons cannot be detected. Similarly, reflected photons are also more likely to pass back through the fluid sample to the dectector, without being absorbed, with the assay device 12 of the present invention. More photons are therefore available for detection.

Use of the assay device 10 of the present invention in conjunction with reflecting the photons emitted by the reaction away from the PMT, towards the PMT, may nearly double the number of detected photons, improving the sensitivity of the measurement. Since the various losses discussed above may be reduced, smaller fluid samples requiring less reagent may be used. For example, a 10 microliter sample may be used with the assay device 10 of the present invention while typical prior art systems require at least 100 microliters.

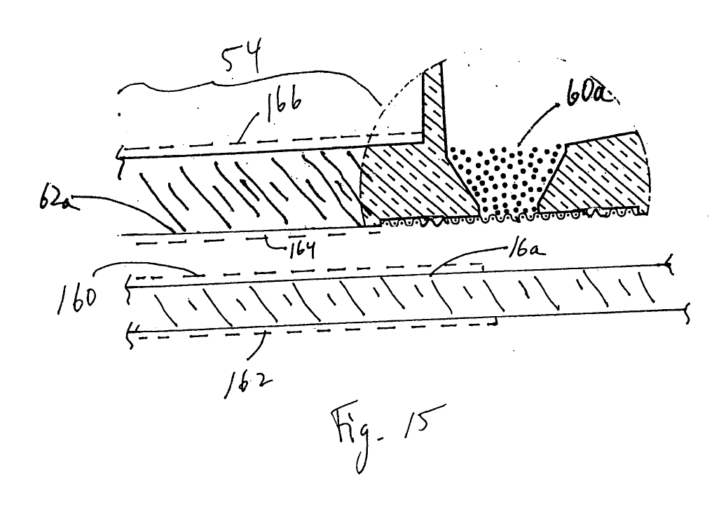

Instead of providing the reflective surface 154 on the supporting surface 156 of the luminometer 150, as in FIG. 14, a reflective surface may be provided on the major planar wall 16a of the base 16, as indicated by phantom line 160 in FIG. 15. A reflective surface can also be provided on the underside of the base 11, below the reading portion, as indicated by the phantom line 162. The reflective surface can also be provided on either the underside 62a of the planar wall 62, as indicated by phantom line 164, or on the reading portion 54, as indicated by the phantom line 16b, if the detector is below the base.

Preferably, the reflective surface is smooth. The reflective surface may also be semi-circular or semi-cylindrical, to focus the reflected light along a point or a line, respectively. A recess (not shown) may be provided in the wall 156 of the luminometer to accommodate a semi-circular or semi-cylindrical reflective surface.

In the embodiments of FIGS. 14-15, a suitably sized strip of aluminum foil could be taped to the wall 156 of the luminometer 150, to the underside of the base 11 or to the reading portion 54. To coat one of the interior surfaces of the chamber 13 with a reflecting material, a metal, such as gold or nickel, could be sputtered or electrodeposited onto the surface, to a thickness of from about 20 Angstroms to about 100 Angstroms.

Luciferin/luciferase reagents are commercially available. An Adenosine 5′ Triphosphate (ATP) Bioluminescent Assay Kit, FL-AAM, available from the Sigma-Aldrich Corporation, Milwaukee, Wis., USA, may be used, for example. FL-AAM is a lyophilized powder containing firefly luciferase, luciferin, MgSO₄, EDTA, dithiothreitol (“DTT”) and bovine serum albumin (“BSA”) in a tricine buffer.

As mentioned above, the assay device 10 of the present invention can also be used in absorption spectrophotometric measurements on fluid samples exposed to assays, where the amount of light absorbed by the fluid sample at a particular wavelength is proportional to the concentration of an analyte in the sample. The intensity of the light emitted by a source may be compared to the intensity of light passing through the fluid sample to determine the absorption of the fluid sample of a particular wavelength, as is known in the art.

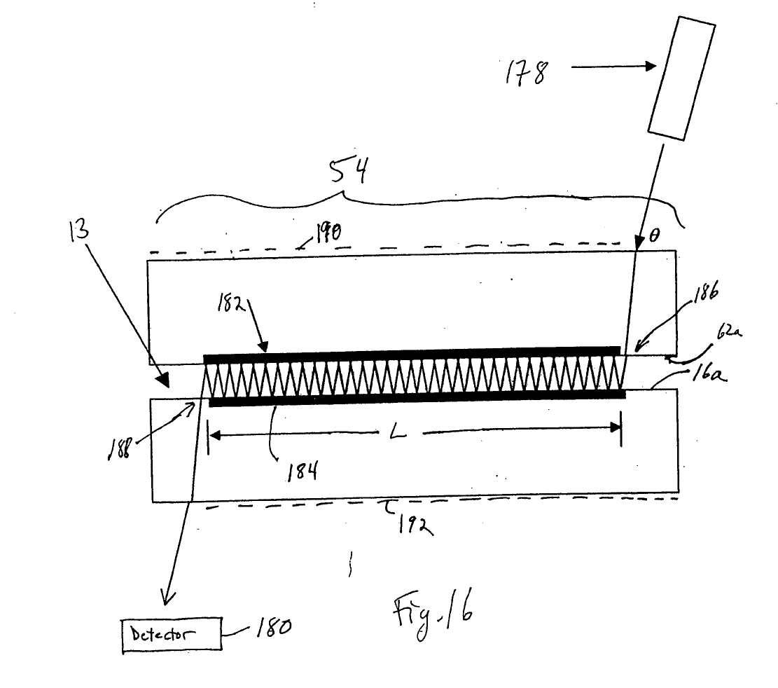

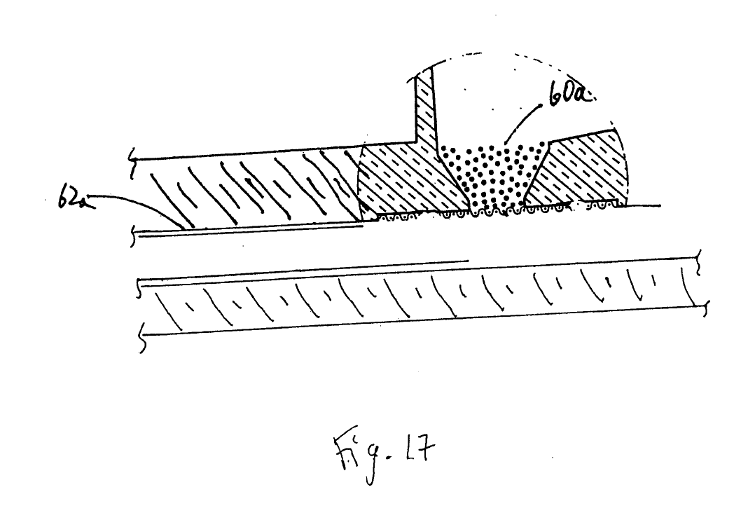

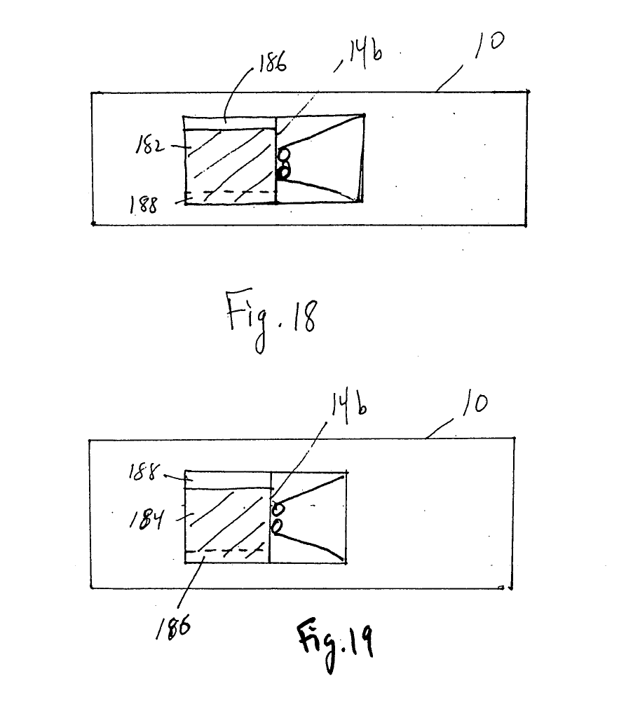

Absorption spectrophotometric measurements may be improved by increasing the length of the optical path through a sample. To increase the optical path length through the fluid sample in the assay device 10 of the present invention, two layers of reflective material are preferably provided, one on each side of the fluid sample. FIG. 16 is a schematic, cross-sectional view of the chamber 13 of the present invention, showing the reading portion 54, the underside 62a of the planar wall 62 and the major bottom wall 16a. A radiation source 170 and a detector 180 are also shown on opposite sides of the capillary chamber 13. Reflective surfaces 182, 184 are shown on the opposing surfaces of the capillary chamber 62a, 16a. A portion of the underside 62a is not covered by the reflective surface 182, to provide an inlet 186 for the radiation into the capillary chamber 13. A portion of the bottom wall 16a of the base is also not covered by the reflective surface 184, to provide an outlet 188 for the radiation exiting the capillary chamber 13. FIG. 17 is a partial cross-sectional view of the assay device 10, showing the reflective surfaces 182, 184.

FIG. 18 and FIG. 19 are top and bottom views of the assay device 10, showing the reflective surfaces 182, 184, the inlet 186 and the outlet 188. In FIG. 17, the outlet 188 is shown in phantom. In FIG. 18, the inlet is shown in phantom. As shown, the inlet 186 and the outlet 188 are preferably rectangular, and extend across the chamber. 13. While the inlet and outlet are shown with longitudinal axes perpendicular to the side wall 14b, they may be oriented parallel to the side wall 14b, as well. While preferred, the inlet 186 and the outlet 188 need not be rectangular. The inlet 186 and the outlet 188, and the source 170 and the detector 180, may be on opposite sides of the assay device 10, as shown in FIG. 16, or may be on the same side of the assay device.

The optical path comprises a plurality of multiple reflections between the opposing reflective surfaces 182, 184 or 190, 192, as shown in FIG. 16. By adjusting the angle θ of the incident light beam with respect to the surface of the assay device, the number of reflections, and hence the effective optical path length R of the light passing through the chamber 13, may be adjusted. The optical path length R=L sec θ, where “L” is the axial length from the inlet 186 to the outlet 188.

Preferably, the number of reflections are adjusted so that the absorption is within a range of from about 0.05 Absorbance Units to about 3.0 Absorbance Units, depending on the absorption coefficient of the analyte, as is known in the art. Lesser absorbance is typically difficult to detect. As absorbance increases above 3.0 Absorbance Units, the relationship between absorbance and analyte concentration is less linear, making the test results difficult to analyze.

The reflective surfaces may be formed of gold or nickel, for example, sputtered or electrodeposited on the desired surface. A thickness from about 20 Angstroms to about 100 Angstroms may be applied. Alternatively, one or both of the reflective surfaces may be provided on an exterior surface of the reading portion 54 and the base, as indicated by phantom lines 190, 192 in FIG. 17. One reflective surface may be within the capillary chamber 13 and the other may be outside of the chamber 13, as well.

The incident light source 178 could be a laser emitting radiation at the desired wavelength band. A polychromatic radiation source may also be used with a filter for filtering out radiation with wavelengths outside of the desired band.

The laser could be mounted on a stand proximate the inlet 186 to the assay device 10. To adjust the number of reflections, the angle θ of the laser with respect to the inlet 186 of the assay device 10 may be varied.

The detector may be a photoconductive device such as a photodiode or CCD, for example. The detector may be mounted proximate the exit of the reading portion to detect the radiation passing through the capillary chamber and out of the outlet 188 of the reading portion 54. Alternatively, the source 178 and detector 180 may be integrated into a reader unit, adapted to receive the assay device. The reflective surfaces could also be provided on opposing surfaces of the reader. The capillary chamber 13 of the assay device 10 may then be inserted between the reflective surfaces. The angle of the incident radiation could be readily varied within the reader.

While reference is made to the capillary chamber 13 of the assay device 10, the chamber receiving the fluid sample need not be a capillary chamber in the embodiment of FIGS. 14-19.

The assay device 10 of the present invention is particularly suited to point-of-care diagnosis and the small sample sizes required for the assays permit multiple and ongoing determinations. Data obtained by the methods and devices of the present invention from patients may also be accumulated in one or more databases to provide a resource for diagnosis and prognosis. A database may also be created for each individual patient, based on numerous measurements taken over a relatively short period of time. In addition, the data obtained for multiple patients can be used to track the initiation and development of disease conditions.

Tables of standard values can also be constructed based on the known values of parameters in the target patient group. Once the table of standard values is constructed, data is collected from a patient on a regular basis and patient-specific databases constructed based on the patient's medical history, current health and the test results. These databases can be used in the development of neural network algorithms, for assessment of current patient test results and diagnoses as well as for predicting certain health outcomes for a given individual.

These applications are described in more detail in WO00/78917, WO00/29847 and U.S. Ser. No. 09/335,732, noted above and incorporated by reference herein, in their entireties.

The assay device 10 of the present invention is also particularly useful in testing food for harmful bacteria and other organisms. Fluid samples from ground beef, beef, cheese, milk, yogurt, juice and chicken, for example, may be analyzed with the assay device 10 of the present invention. Other food products, as well as samples from food preparation surfaces in restaurants and salad bars, for example, may be analyzed, as well.

The assay device of the present invention is also particularly useful in medical research that employs experimental animals, such as rodents. By requiring smaller amounts of bodily samples from each individual animal to conduct an assay, the assay device of the present invention makes it possible to take multiple samples from the animal over short periods of time. Fewer animals may therefore be needed for each experiment, since multiple samples may be taken from the same animal at different times.

It is understood that changes may be made to the embodiments described above, without departing from the scope of the present invention, which is defined by the following claims.

We claim:

1. An assay device comprising: a base comprising a major wall and at least one side wall transverse to the major wall, the side wall and the major wall defining a cavity; and an insert received in the cavity, the insert comprising: first and second opposed surfaces, the first surface comprising a portion opposing the major wall, the portion of the first surface and the major wall being separated to define a space and the first surface of the insert and the major wall defining an entrance to the space; the second surface of the insert defining an input portion for receipt of a fluid sample, the input portion comprising: a passage defined by the insert, the passage extending through the insert, the passage having an entrance in the input portion and an exit in the first surface, the exit being proximate to the entrance to the space; a plurality of particles supported within the passage, wherein, when a fluid sample passes through the passage, the particles are transiently abutting, defining transient interstitial spaces therebetween, to filter materials greater than a predetermined size from the fluid sample; and the second surface of the insert further defining a reading portion for analyzing the sample drawn into the space.

2. The assay device of claim 1, further comprising a porous material attached to the first surface of the insert, over the exit, to support the particles.

3. The assay device of claim 2, wherein the porous membrane is chosen from the group consisting of a nylon mesh, a polyester mesh, a polycarbonate film and a polysulfone membrane.

4. The device of claim 1, further comprising a reagent associated with the particles.

5. The assay device of claim 3, wherein the reagent comprises a label specific to an analyte.

6. The assay device of claim 5, wherein the label is a fluorescent label, a radioactive label or a metal label.

7. The assay device of claim 1, further comprising a plurality of second particles having a size less than the size of the plurality of first particles.

8. The assay device of claim 7, further comprising a reagent associated with the second particles.

9. The assay device of claim 1, further comprising a reagent in the space.

10. The assay device of claim 1, wherein the particles are chosen from the group consisting of latex, glass, silicon and sand.

11. The assay device of claim 1, wherein the cavity is recessed in the base.

12. The assay device of claim 1, wherein the side wall extends from the base.

13. The assay device of claim 1, wherein the distance between the portion and the major wall is effective to cause capillary flow of a fluid sample at the entrance into the space.

14. An assay device comprising: a base defining an enclosed cavity, the cavity having a major surface and an open face opposite the major surface; and a plate within the cavity, the plate having a first surface and a second surface opposed to the first surface, the second surface opposing the major surface; wherein a portion of the second surface is separated from the major surface to define a space, and the insert and the major surface of the cavity define an entrance to the space; and the first surface of the plate defines an input portion for the receipt of a fluid sample, and a reading portion for viewing fluid sample drawn into the space.

15. The assay device of claim 14, wherein the cavity is recessed in the base.

16. The assay device of claim 14, further comprising a side wall extending transverse to the base and surrounding the major surface, defining the cavity.

17. The assay device of claim 14, wherein the insert defines at least one passage from the input portion to the entrance to the space.

18. The assay device of claim 14, further comprising a plurality of particles in the at least one passage, the particles being transiently abutting when a fluid passes through the particles, the transiently abutting particles defining transient interstitial spaces therebetween, the particles removing materials greater than a predetermined size from the fluid sample.

19. The assay device of claim 14, further comprising a reagent in the space.

20. The assay device of claim 14, wherein the portion of the second surface and the major surface are separated by a distance such that fluid proximate the entrance is drawn into the space by capillary force.

21. An assay device comprising: a base comprising a major wall and a side wall transverse to the major wall, the side wall and major wall defining a cavity; and an insert press-fit in the cavity, the insert comprising: first and second opposed surfaces, the second surface comprising a portion opposing the major wall, the portion of the second surface and the major wall having a space therebetween; the first surface of the insert defining an input portion for receipt of a fluid sample, the input portion being in fluid communication with the space; and a reading portion for analyzing fluid sample drawn into the space.

22. The assay device of claim 21, wherein the side wall has an interior surface comprising a plurality of protrusions engaging a side edge of the insert in the press-fit.

23. The assay device of claim 22, further comprising a plurality of second protrusions from the side edge of the insert, the locations of the plurality of second protrusions corresponding to the locations of the plurality of first protrusions on the side wall, so that the first protrusions bear against the second protrusions when the insert is press-fit within the chamber.

24. The assay device of claim 21, wherein the insert has a side edge comprising a plurality of protrusions engaging an interior surface of the side wall.

25. The assay device of claim 21, further comprising a plurality of second protrusions protruding from the interior surface of the side wall, the locations of the plurality of second protrusions corresponding to the locations of the plurality of first protrusions on the side wall, so that the first protrusions bear against the second protrusions when the insert is press-fit within the chamber.

26. The assay device of claim 21, further comprising a reagent within the space.

27. The assay device of claim 21, wherein the side wall extends transverse to the base and surrounds the major wall, defining the cavity.

28. The assay device of claim 21, wherein the insert defines at least one passage through the insert, providing fluid communication from the input portion to the space.

29. The assay device of claim 28, further comprising a plurality of particles in the at least one passage, the particles being transiently abutting when a fluid passes through the particles, the transiently abutting particles defining transient interstitial spaces therebetween, the particles removing materials greater than a predetermined size from the fluid sample.

30. The assay device of claim 21, wherein the portion of the surface and the major wall are separated by a distance effective to cause capillary flow of a fluid sample into the space.

31. An assay device comprising: a base defining an enclosed cavity, the cavity having a major surface and an open face opposite the major surface; and a plate within the cavity, the plate having a first surface and a second surface, the first surface opposing the major surface; wherein a portion of the first surface is separated from the major surface to define a space, and the insert and the surface of the cavity define an entrance to the space; the first surface of the plate defining an input portion for the receipt of a fluid sample; and a reading portion for analyzing fluid sample drawn into the space; the assay device further comprising a lid slideably engaging the base, such that the lid may be selectively positioned over the input portion.

32. The assay device of claim 31, wherein the base defines a pair of grooves for engaging the lid.

33. The assay device of claim 32, wherein the lid comprises: a major portion for selectively covering the input portion, the major portion having two opposing ends;
two arm portions, each having a first end depending from a respective opposing end of the major portion, and a second end; and inwardly directed flanges depending from the second end of each arm, each flange engaging a respective groove.

34. The assay device of claim 33, wherein the side wall has a top edge, and at least one protrusion protruding from the top edge of the side wall adjacent to the input portion, such that when the lid is moved over the protrusion, the lid is locked in place.

35. The assay device of claim 31, wherein the lid may be selectively positioned over the reading portion.

36. An assay device comprising: a base defining an enclosed cavity, the cavity having a major surface and an open face opposite the major surface; and a plate within the cavity, the plate having a first surface and a second surface, the first surface opposing the major surface; wherein a portion of the first surface is separated from the major surface to define a space, and the first surface and the surface of the cavity define an entrance to the space; the first surface of the plate defining an input portion for the receipt of a fluid sample, the insert defining at least one passage through the insert, providing fluid communication from the input portion to the entrance to the space; the input portion further comprising a wall extending transverse to the input portion, the wall surrounding the input portion, and the second surface of the plate further defining a reading portion for analyzing fluid sample drawn into the space.

37. The assay device of claim 36, wherein the input portion comprises at least one surface tapered towards the passage.

38. The assay device of claim 37, wherein the input portion comprises three surfaces tapered towards the passage.

39. The assay device of claim 36, comprising two adjacent passages.

40. The assay device of claim 36, wherein a portion of the wall is adjacent to the passage.

41. The assay device of claim 36, wherein the cavity is recessed in the base.

42. The assay device of claim 36, further comprising a side wall extending transverse to the base and surrounding the major surface, defining the cavity.

43. The assay device of claim 36, further comprising a plurality of particles in the at least one passage, the particles being transiently abutting when a fluid sample passes through the particles, the transiently abutting particles defining transient interstitial spaces therebetween, the particles removing material greater than a predetermined size from the fluid sample.

44. The assay device of claim 36, further comprising a reagent in the space.

45. The assay device of claim 36, wherein the portion of the first surface is separated from the major surface by a distance effective to cause capillary flow of fluid sample at the entrance, into the space.

46. An assay device comprising: a base comprising a major wall and a side wall transverse to the major wall, the side wall surrounding the major wall to define a cavity with an open face opposite the major wall; and an insert for being received in the cavity, the insert comprising: first and second opposed surfaces, the first surface comprising a portion opposing the major wall when the insert is received in the cavity, the portion and the major wall being separated to define a space and the first surface of the insert and the major wall defining an entrance to the space; the second surface of the insert defining: an input portion for receipt of the fluid sample, the input portion being in fluid communication with the entrance to the space, and a reading portion for analyzing a fluid sample in the space, the reading portion opposing the space.

47. The assay device of claim 46, further comprising a multi-layer carrier in the space.

48. The assay device of claim 47, wherein the multi-layer carrier comprises a dry matrix layer and a gel layer adjacent to the dry matrix layer.

49. The assay device of claim 48, wherein at least one of the two layers comprises a reagent.

50. The assay device of claim 48, wherein each layer comprises a different reagent.

51. The assay device of claim 47, comprising alternating dry matrix layers and gel layers.

52. The assay device of claim 48, wherein the dry matrix layer comprises cellulose.

53. The assay device of claim 46, further comprising a layer of gel in the space.

54. The assay device of claim 52, wherein the gel layer comprises agar.

55. An assay device comprising: a base defining an enclosed cavity, the cavity having a major surface and an open face opposite the major surface; a plate within the cavity, the plate having a first surface and a second surface opposed to the first surface, the first surface opposing the major surface of the cavity; wherein a portion of the first surface is separated from the major surface to define a space, and the insert and the major surface of the cavity define an entrance to the space; and the second surface of the plate defines an input portion for the receipt of a fluid sample, and a second portion opposing the space; and the assay device further comprising a pair of opposing reflective surfaces, on opposite sides of the space.

56. The assay device of claim 55, wherein the first reflective surface is on the first surface of the plate and the second reflective surface is on the major surface of the cavity.

57. The assay device of claim 56, wherein one of the reflective surfaces defines an inlet for radiation into the space and the other reflective surface defines an outlet for radiation out of the space, the inlet and the outlet being positioned with respect to each other such that radiation entering the space through the inlet is reflected multiple times between the reflective surfaces prior to exiting the space.

58. The assay device of claim 56, wherein the first reflective surface is on the second portion of the second surface and the second reflective surface is on an underside of the base.

59. The assay device of claim 21, further comprising protrusions on the portion of the first surface opposing the major wall, the protrusions engaging the major wall.

60. The assay device of claim 59, wherein the protrusions extend longitudinally extending across the portion.

61. The assay device of claim 21, further comprising protrusions on the major wall, the protrusions engaging the portion of the first surface.

62. The assay device of claim 61, wherein the protrusions extend longitudinally across the major wall.

63. The assay device of claim 21, further comprising means for supporting the portion of the first surface opposing the major wall, a predetermined distance from the major wall, said means being between the portion and the major wall.
